(19) United States
(12) Patent Application Publication
(10) Pub. No.: US 2004/0043386 A1
(43) Pub. Date: Mar. 4, 2004

(54) Methods and compositions for functional ubiquitin assays

(76) Inventors: Todd Pray, San Francisco, CA (US); Brian Wong, Los Altos, CA (US); Mark Bennett, Moraga, CA (US); Frank Parlati, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/232,951

(22) Filed: Aug. 30, 2002

(51) Int. Cl. C12Q 1/68; G01N 33/53; G01N 33/567; C12N 15/867

(52) U.S. Cl. 435/6; 435/7.2; 435/456

(57) ABSTRACT

The present attention is directed to compositions and methods for performing functional assays to determine the physiological role of ubiquitin agents and ubiquitin moieties.

### FIELD OF THE INVENTION

The present attention is directed to compositions and methods for performing functional assays to determine the physiological role of ubiquitin agents and ubiquitin moieties.

### BACKGROUND OF THE INVENTION

Ubiquitin is a highly conserved 76 amino acid protein expressed in all eukaryotic cells. The levels of many intracellular proteins are regulated by a ubiquitin-mediated proteolytic process. This process involves the covalent ligation of ubiquitin to a target protein, resulting in a poly-ubiquitinated target protein which is rapidly detected and degraded by the 26S proteasome.

The ubiquitination of these target proteins is known to be mediated by the enzymatic activity of three ubiquitin agents. Ubiquitin is first activated in an ATP-dependent manner by a ubiquitin activating agent, for example, an E1. The C-terminus of a ubiquitin forms a high energy thiolester bond with the ubiquitin activating agent. The ubiquitin is then transferred to a ubiquitin conjugating agent, for example, an E2 (also called ubiquitin moiety carrier protein), also linked to this second ubiquitin agent via a thiolester bond. The ubiquitin is finally linked to its target protein (e.g. substrate) to form a terminal isopeptide bond under the guidance of a ubiquitin ligating agent, for example, an E3. In this process, monomers or oligomers of ubiquitin are attached to the target protein. On the target protein, each ubiquitin is covalently ligated to the next ubiquitin through the activity of a ubiquitin ligating agent to form polymers of ubiquitin.

The enzymatic components of the ubiquitination pathway have received considerable attention (for a review, see Weissman, Nature Reviews 2:169-178 (2001)). The members of the E1 ubiquitin activating agents and E2 ubiquitin conjugating agents are structurally related and well characterized enzymes. There are numerous species of E2 ubiquitin conjugating agents, some of which act in preferred pairs with specific E3 ubiquitin ligating agents to confer specificity for different target proteins. While the nomenclature for the E2 ubiquitin conjugating agents is not standardized across species, investigators in the field have addressed this issue and the skilled artisan can readily identify various E2 ubiquitin conjugating agents, as well as species homologues (See Haas and Siepmann, FASEB J. 11:1257-1268 (1997)).

Generally, ubiquitin ligating agents contain two separate activities: a ubiquitin ligase activity to attach, via an isopeptide bond, monomers or oligomers of ubiquitin to a target protein, and a targeting activity to physically bring the ligase and substrate together. The substrate specificity of different ubiquitin ligating agents is a major determinant in the selectivity of the ubiquitin-mediated protein degradation process.

In eukaryotes, some ubiquitin ligating agents contain multiple subunits that form a complex called the SCF having ubiquitin ligating activity. SCFs play an important role in regulating Gl progression, and consists of at least three subunits, SKP1, Cullins (having at least seven family members) and an Fbox protein (of which hundreds of species are known) which bind directly to and recruit the substrate to the complex. The combinatorial interactions between the SCF's and a recently discovered family of RING finger proteins, the ROC/APC11 proteins, have been shown to be the key elements conferring ligase activity to ubiquitin ligating agents. Particular ROC/Cullin combinations can regulate specific cellular pathways, as exemplified by the function of APC11-APC2, involved in the proteolytic control of sister chromatid separation and exit from telophase into G1 in mitosis (see King et al., supra; Koepp et al., Cell 97:431-34 (1999)), and ROC1-Cullin 1, involved in the proteolytic degradation of IKB in NF-KB/IKB mediated transcription regulation (Tan et al., Mol. Cell 3(4):527-533 (1999); Laney et al., Cell 97:427-30 (1999)).

The best characterized ubiquitin ligating agent is the APC (anaphase promoting complex), which is multi-component complex that is required for both entry into anaphase as well as exit from mitosis (see King et al., Science 274:1652-59 (1996) for review). The APC plays a crucial role in regulating the passage of cells through anaphase by promoting ubiquitin-mediated proteolysis of many proteins. In addition to degrading the mitotic B-type cyclin for inactivation of CDC2 kinase activity, the APC is also required for degradation of other proteins for sister chromatid separation and spindle disassembly. Most proteins known to be degraded by the APC contain a conserved nine amino acid motif known as the “destruction box” that targets them for ubiquitin ubiquitination and subsequent degradation. However, proteins that are degraded during G1, including G1 cyclins, CDK inhibitors, transcription factors and signaling intermediates, do not contain this conserved amino acid motif. Instead, substrate phosphorylation appears to play an important role in targeting their interaction with a ubiquitin ligating agent for ubiquitin ubiquitination (see Hershko et al., Ann. Rev. Biochem. 67:429-75 (1998)).

Two major classes of E3 ubiquitin ligating agents are known: the HECT (homologous to E6-AP carboxy terminus) domain E3 ligating agents; and the RING finger domain E3 ligating agents. E6AP is the prototype for the HECT domain subclass of E3 ligating agents and is a multi-subunit complex that functions as a ubiquitin ligating agent for the tumor suppressor p53 which is activated by papillomavirus in cervical cancer (Huang et al. (1999) Science 286:1321-1326). Members of this class are homologous to the carboxyl terminus of E6AP and utilize a Cys active site to form a thiolester bond with ubiquitin, analogous to the E1 activating agents and E2 conjugating agents. However, in contrast, the members of the RING finger domain class of E3 ligating agents are thought to interact with an ubiquitin-conjugated-E2 intermediate to activate the complex for the transfer of ubiquitin to an acceptor. Examples of the RING domain class of E3 ligating agents are TRAF6, involved in IKK activation; Cbl, which targets insulin and EGF; Sina/Siah, which targets DCC; Itchy, which is involved in haematopoesis (B, T and mast cells); IAP, involved with inhibitors of apoptosis; and Mdm2 which is involved in the regulation of p53.

The RING finger domain subclass of E3 ligating agents can be further grouped into two subclasses. In one subclass, the RING finger domain and the substrate recognition domain are contained on different subunits of a complex forming the ubiquitin ligating agent (e.g., the RBx1 and the F-box subunit of the SCF complex). In the second subclass of ubiquitin ligating agents, the ligating agents have the RING finger domain and substrate recognition domain on a single subunit. (e.g., Mdm2 and cbl) (Tyers et al. (1999) Science 284:601, 603-604; Joazeiro et al. (2000) 102:549-552). A further class of ligating agents are those having a “PHD” domain and are homologs of the RING finger domain ligating agents (Coscoy et al. (2001) J. Cell Biol. 155(7):1265-1273), e.g., MEKK1. The PHD domain ligating agents are a novel class of membrane-bound E3 ligating agents.

In addition, a new class of ubiquitin ligases have been characterized. These are the U-box-containing proteins. (Patterson, Sci STKE 2002(116:PE4 (220)). This class, for the present, represents a small number of ligases which have yet to be extensively characterized.

Mdm2 belongs to the second subclass of single subunit E3 ligating agents and is involved in regulating the function and stability of p53, an important tumor suppressor. In cells, p53 functions as a DNA-binding transcription factor which induces the expression of genes involved in DNA repair, apoptosis, and the arrest of cell growth. In approximately 50% of all human cancer p53 is inactivate by deletion or mutation. The level of p53 in the cell is maintained at low steady-state levels, and is induced and activated post-translationally by various signal pathways responsive to cellular stress (Lakin et al. (1999) Oncogene 18:7644-7655; Oren, M. (1999) J. Biol. Chem 274:36031-36,034). Stimuli that trigger the stress response and activate p53 include oxygen stress, inappropriate activation of oncogenes and agents that cause damage to DNA (e.g., ionizing radiation, chemicals, and ultra violet light).

The carboxyl terminus of Mdm2 contains a variant of the RING finger domain (Saurin et al. (1996) Trends Biochem. Sci. 21:208-214) that is critical for the activity of this E3 ligating agent. Recent studies have shown that Mdm2 mediates the ubiquitination of itself resulting in the formation of poly-ubiquitin chains on the protein (Zhihong et al. (2001) J.B.C. 276:31,357-31,367; Honda et al. (2000) Oncogene 19:1473-1476; Shengyun et al. (2000) 275:8945-8951). Further, the ubiquitin ligating activity of Mdm2 is dependent on its RING finger domain.

Typically, the ubiquitination of target proteins by E3 in cells results in the formation of poly-ubiquitin chains. An isopeptide bond is formed between the carboxyl terminus of the ubiquitin and the ε-amino group of Lys in the target protein. The extension or formation of ubiquitin chains results from the formation of additional isopeptide bonds with the Lys⁴⁸ (and sometimes Lys⁶³) of a previously conjugated ubiquitin and the carboxyl-terminal Gly of an additional ubiquitin. The efficient recognition of a ubiquitinated target protein by a proteosome requires at least four ubiquitins linked in this configuration. However, in the case of Mdm2-mediated ubiquitination of p53, neither Lys⁴⁸ or Lys⁶³ is involved in the formation of poly-ubiquitin chains. Recent studies show that human Mdm2 mediates multiple mono-ubiquitination of p53 by a mechanism requiring enzyme isomerization (Zhihong et al. (2001) J.Biol.Chem. 276:31,357-31,367). Further, in vitro, the transfer of ubiquitin to p53 can occur independent of E1 when using an E2 pre-conjugated with ubiquitin. These results suggest that the pre-conjugated E2 can bind to Mdm2 and thereafter transfer the ubiquitin to the Mdm2 in the absence of an E1.

Thus, ubiquitin agents, such as the ubiquitin activating agents, ubiquitin conjugating agents, and ubiquitin ligating agents, are key determinants of the ubiquitin-mediated proteolytic pathway that results in the degradation of targeted proteins and regulation of cellular processes. Consequently, agents that modulate the activity of such ubiquitin agents may be used to upregulate or downregulate specific molecules involved in cellular signal transduction. Disease processes can be treated by such up- or down regulation of signal transducers to enhance or dampen specific cellular responses. This principle has been used in the design of a number of therapeutics, including phosphodiesterase inhibitors for airway disease and vascular insufficiency, kinase inhibitors for malignant transformation and Proteasome inhibitors for inflammatory conditions such as arthritis.

Due to the importance of ubiquitin-mediated proteolysis in cellular process, for example cell cycle regulation, there is a need for a fast and simple means for identifying the physiological role of ubiquitin agents that are catalytic components of this enzymatic pathway, and for identifying which ubiquitin agents are involved in various regulatory pathways. Thus, an object of the present invention is to provide methods of assaying for the physiological role of ubiquitin agents, and for providing methods for determining which ubiquitin agents are involved together in a variety of different physiological pathways.

### SUMMARY OF THE INVENTION

In accordance with the objects outlined above, the present invention provides a method comprising providing a library of cells comprising a library of nucleic acids comprising nucleic acid encoding at least one variant ubiquitin agent selected from the group consisting of ubiquitin activating agents, ubiquitin conjugating agents and ubiquitin ligating agents, screening the library of cells for an altered phenotype as compared to control cells, isolating at least one altered cell with the altered phenotype; and identifying the variant agent in the altered cell.

In addition, the invention provides a method comprising providing a cell culture, introducing into cells of said cell culture a library of nucleic acids comprising nucleic acids encoding variants of ubiquitin activating, ubiquitin conjugating or ubiquitin ligating agents, or antisense or siRNA directed to ubiquitin activating, ubiquitin conjugating or ubiquitin ligating agents, screening said cell cultures for altered phenotype as compared to control cells, and identifying the dominant negative mutant ubiquitin activating, ubiquitin conjugating or ubiquitin ligating agent, antisense or siRNA that caused said altered phenotype.

In addition, the invention provides a method for determining which ubiquitin agents are involved together in a given signal transduction or physiological pathway. The method involves providing in a combinatorial fashion, a ubiquitin ligating agent, a ubiquitin activating agent, and a ubiquitin conjugating agent and a plurality of cell cultures, and screening the cell cultures for an effect in a physiological pathway or functional assay.

### BRIEF DESCRIPTION OF THE DRAWINGS

FIG. 1 depicts the amino acid sequence of human ubiquitin.

FIG. 2 depicts a flowchart of the procedure for the ICAM assay.

FIGS. 3A and 3B show the nucleic acid sequence and amino acid sequence, respectively, of a human E1, Uba1 (E1).

FIGS. 4A and 4B show the nucleic acid sequence and amino acid sequence, respectively, of a human E1, Uba3 homolog.

FIGS. 5A and 5B show the nucleic acid sequence and amino acid sequence, respectively, of a human E1,SAE1.

FIGS. 6A and 6B show the nucleic acid sequence and amino acid sequence, respectively, of a human E1, UBE1L.

FIGS. 7A and 7B show the nucleic acid sequence and amino acid sequence, respectively, of a human E1, APG7 isoform.

FIGS. 8A and 8B show the nucleic acid sequence and amino acid sequence, respectively, of a human E1, FLJ14657.

FIGS. 9A and 9B show the nucleic acid sequence and amino acid sequence, respectively, of a human E2, FTS.

FIGS. 10A and 10B show the nucleic acid sequence and amino acid sequence, respectively, of a human E2, XM_{—}054332.

FIGS. 11A and 11B show the nucleic acid sequence and amino acid sequence, respectively, of a human E2, Ubc8.

FIGS. 12A and 12B show the nucleic acid sequence and amino acid sequence, respectively, of a human E2, UbcH9.

FIGS. 13A and 13B show the nucleic acid sequence and amino acid sequence, respectively, of a human E2, Ubc12.

FIGS. 14A and 14B show the nucleic acid sequence and amino acid sequence, respectively, of a human E2, MGC10481.

FIGS. 15A and 15B show the nucleic acid sequence and amino acid sequence, respectively, of a human E2, UbcH6.

FIGS. 16A and 16B show the nucleic acid sequence and amino acid sequence, respectively, of a human E2, HIP2.

FIGS. 17A and 17B show the nucleic acid sequence and amino acid sequence, respectively, of a human E2, Uev1.

FIGS. 18A and 18B show the nucleic acid sequence and amino acid sequence, respectively, of a human E2, Ubc13.

FIGS. 19A and 19B show the nucleic acid sequence and amino acid sequence, respectively, of a human E3,MDM2.

### DETAILED DESCRIPTION OF THE INVENTION

Ubiquitination is becoming appreciated as one of the more important post translational modifications within a cell. Various molecules involved with ubiquitination have been discovered. However, the physiological role of these molecules remains unclear. That is, while a variety of molecules involved in ubiquitination have been discovered, their specific physiological roles are unknown.

In addition, there is an increasingly appreciated population of ubiquitin-like molecules whose role remains unclear, as well. That is, these molecules, which resemble ubiquitin, presumably are involved in a multitude different physiological processes, however it is unclear which ones. Thus, to elucidate the physiological role of the variety of ubiquitin-like molecules and ubiquitin modulating molecules (ubiquitin agents) remains a significant task.

Moreover, as the number of ubiquitin modulating molecules increases, their role in signal transduction and cellular regulation becomes increasingly complex. Thus, there is a need for method to elucidate the combinatorial relationships between the different ubiquitin modulating molecules. That is, there is a need to identify which ubiquitin modulating molecules are involved with and regulate a particular signal transduction pathway or are involved in a specific physiological process.

Accordingly, the present invention provides a method for performing functional ubiquitination screens. The methods include providing a cell culture, whose cells contain a library of nucleic acids comprising nucleic acids encoding variant ubiquitin agents such as ubiquitin activating, ubiquitin conjugating or ubiquitin ligating agents. The invention further provides screening the cell culture for altered phenotype as compared to control cells, isolating those with altered phenotypes and identifying the variant ubiquitin agent(s) that resulted in the altered phenotype.

In one embodiment, the invention provides culturing cells expressing different ubiquitin agents and assaying a functional readout for the activity of the ubiquitin agents. Modulation of the functional assay indicates involvement of the ubiquitin agent in that pathway.

By “ubiquitin agents” is meant a molecule involved in ubiquitination, most frequently enzymes. Ubiquitin agents can include ubiquitin activating agents, ubiquitin ligating agents and ubiquitin conjugating agents. In addition, ubiquitin agents can include ubiquitin moieties as described below. In addition, deubiquitination agents (e.g. proteases that degrade or cleave ubiquitin or polyubiquitin chains) find use in the invention.

As noted previously, examples of ubiquitin agents are ubiquitin activating agents, ubiquitin conjugating agents, and ubiquitin ligating agents. In preferred embodiments, the ubiquitin activating agent is preferably an E1 or a variant thereof; the ubiquitin conjugating agent is preferably an E2 or a variant thereof; and the ubiquitin ligating agent is preferably an E3 or variant thereof. Thus, the present invention provides methods for determining the physiological role of ubiquitin activating agents, ubiquitin conjugating agents, ubiquitin ligating agents, and ubiquitin moieties, either individually or in combination. In addition, the present invention provides methods of assaying for agents that modulate the attachment of a ubiquitin moiety to a ubiquitin agent, target protein, or mono- or poly-ubiquitin moiety preferably attached to a ubiquitin agent or target protein.

In general, the methods involve expressing a ubiquitin moiety and one or more ubiquitin agents in a cell system and determining the effect of the ubiquitin moiety, ubiquitin agent or variant of the ubiquitin moiety or ubiquitin agent in a functional assay. The functional assay may involve a cellular readout as described below, or may involve determining the amount of ubiquitin on a target protein. That is, the method involves measuring the amount of ubiquitin moiety attached to at least one of the following substrate molecules: a ubiquitin agent; a target protein; or a mono- or poly-ubiquitin moiety which is preferably attached to a ubiquitin agent or target protein.

Ubiquitin ligase assays are described in more detail in U.S. application Ser. Nos. 09/542,497, filed Apr. 3, 2000; 09/826,312, filed Apr. 3,2001; 10/091,174, filed Mar. 4, 2002; 10/108,767, filed Mar. 26, 2002; 10/152,156, filed May 20, 2002, all of which are expressly incorporated herein by reference. In addition, ubiquitin protease assays are described in U.S. Ser. No. - - - - - -, filed Aug. 30, 2002 (Attorney docket number A-71410), which is expressly incorporated herein by reference.

Accordingly, the present invention provides methods comprising providing a library of cells comprising a library of nucleic acids comprising nucleic acid encoding at least one variant ubiquitin agent. By “cells” herein is meant any prokaryotic or eukaryotic cell. Preferred embodiments use eukaryotic cells, although as will be appreciated by those in the art, the type of cells used in the present invention can vary widely. Appropriate cells include yeast, bacteria, archaebacteria, fungi, and insect and animal cells, including mammalian cells. Of particular interest are Drosophila melanogaster cells, Pichia pastoris and P. methanolica, Saccharomyces cerevisiae and other yeasts, E. coli, Bacillus subtilis, SF9 cells, SF21 cells, C129 cells, Saos-2 cells, Hi-5 cells, 293 cells, Neurospora, BHK, CHO, COS, and HeLa cells. Of greatest interest are A549, HeLa, Jurkat, BJAB, HUVEC, CHMC, HCT116.

When mammalian cells are used, basically, any mammalian cells may be used, with mouse, rat, primate and human cells being particularly preferred. Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH 3T3 cells, CHO, Cos, etc. See the ATCC cell line catalog, hereby expressly incorporated by reference.

By “library” herein is meant a plurality. In a preferred embodiment, the libraries provided herein comprise between about 10 and about 10⁷ independent clones, with from about 10² to about 10⁶ being preferred. In one particularly preferred embodiment, the library is a library of variant ubiquitin agents such as dominant negative ubiquitin agents. That is, the library encodes truncations, and deletions or mutants of ubiquitin agents as described herein. In an alternative embodiment, the library is a library of antisense molecules directed to different ubiquitin agents. Alternatively, the library is a library encoding siRNA directed to various ubiquitin agents.

The cells comprise nucleic acid encoding at least one variant ubiquitin agent. By “nucleic acid” herein is meant either DNA or RNA, or molecules which contain both deoxy- and ribonucleotides. The nucleic acids include genomic DNA, cDNA and oligonucleotides including sense and anti-sense nucleic acids. Also siRNA are included. Such nucleic acids may also contain modifications in the ribose-phosphate backbone to increase stability and half life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”). By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

The nucleic acids encoding at least one variant ubiquitin agent. By “ubiquitin agent” herein is meant ubiquitin activating agent, ubiquitin conjugating agent, ubiquitin ligating agent and ubiquitin moieties, as described above.

As used herein “ubiquitin activating agent” refers to a ubiquitin agent, preferably a protein, capable of transferring or attaching a ubiquitin moiety to a ubiquitin conjugating agent. In a preferred embodiment, the ubiquitin activating agent forms a high energy thiolester bond with ubiquitin moiety, thereby “activating” the ubiquitin moiety. In another preferred embodiment, the ubiquitin activating agent binds or attaches ubiquitin moiety. In another preferred embodiment, the ubiquitin activating agent is capable of transferring or attaching ubiquitin moiety to a substrate molecule that is a monoor poly-ubiquitin moiety. In a preferred embodiment, the ubiquitin activating agent is capable of transferring or attaching ubiquitin moiety to a mono- or poly-ubiquitinated ubiquitin conjugating agent.

In a preferred embodiment the ubiquitin activating agent is an E1. In a preferred embodiment, the E1 is capable of transferring or attaching ubiquitin moiety to an E2, defined below.

In the methods and compositions of the present invention, the ubiquitin activating agent comprises an amino acid sequence or a nucleic acid corresponding to a sequence of an Genbank data base accession number listed in Table 1 below and incorporated herein by reference.

TABLE 1

| | | | ACCESSION |
|---|---|---|---|
| ORG | SYMBOL | DESCRIPTION | NO. |
| Hs | APPBPI | amyloid beta precursor protein binding protein 1, 59 kD | NM_003905 |
| Hs | FLJ23251 | hypothetical protein FLJ23251 | NM_024818 |
| Hs | GSA7 | ubiquitin activating enzyme E1-like protein | NM_006395 |
| Hs | | similar to ubiquitin-activating enzyme E1 (A1S9T and BN75 | XM_088743 |
| | | temperature sensitivity complementing) (H. sapiens) | |
| Hs | | similar to SUMO-1 activating enzyme subunit 1; SUMO-1 | XM_090110 |
| | | activating enzyme E1 N subunit; sentrin/SUMO-activating | |
| | | protein AOS1; ubiquitin-like protein SUMO-1 activating enzyme | |
| Hs | SAE1 | SUMO-1 activating enzyme subunit 1 | NM_005500 |
| | | | and |
| | | | XM_009036 |
| Dm | Uba1 | Ubiquitin activating enzyme 1 | NG_000652 |
| | | | and |
| | | | NM_057962 |
| Dm | Uba2 | Smt3 activating enzyme 2 | NM_080017 |
| Hs | UBA2 | SUMO-1 activating enzyme subunit 2 | NM_005499 |
| Hs | UBE1 | ubiquitin-activating enzyme E1 (A1S9T and BN75 temperature | NM_003334 |
| | | sensitivity complementing) | and |
| | | | XM_033895 |
| Hs | UBE1C | ubiquitin-activating enzyme E1C (UBA3 homolog, yeast) | NM_003968 |
| Rn | Ube1c | Ubiquitin-activating enzyme E1C | NM_057205 |
| Mm | Ube1l | Ubiquitin-activating enzyme E1-like | |
| Hs | UBE1L | Ubiquitin-activating enzyme E1-like | NM_003335 |
| Mm | Ube1x | ubiquitin-activating enzyme E1, Chr X | NM_009457 |
| Mm | Ube1y1 | ubiquitin-activating enzyme E1, Chr Y 1 | NM_011667 |
| Mm | Ube1y1- | ubiquitin-activating enzyme E1, Chr Y, pseudogene 1 | M88481 and |
| | ps1 | | U09053 |
| Mm | Ube1y1- | ubiquitin-activating enzyme E1, Chr Y-1, pseudogene 2 | U09054 |
| | ps2 | | |

Sequences encoding a ubiquitin activating agent may also be used to make variants thereof that are suitable for use in the methods and compositions of the present invention. The ubiquitin activating agents and variants suitable for use in the methods and compositions of the present invention may be made as described herein.

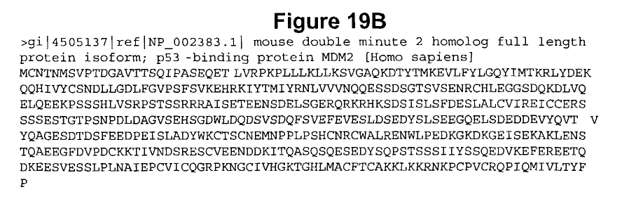

In a preferred embodiment, E1 proteins useful in the invention include the polypeptides comprising sequence disclosed in FIGS. 19-24 or poleptides encoded by nucleic acids having sequences disclosed in the same figures. In other preferred embodiments, the E1 proteins are encoded by nucleic acids comprising the sequences represented by the accession numbers provided in Table 1. In on preferred embodiment, E1 is human E1. E1 is commercially available from Affiniti Research Products (Exeter, U.K.). Variants of the cited E1 proteins, also included in the term “E1”, can be made as described herein.**

In some embodiments, the methods of the present invention comprise the use of a ubiquitin conjugating agent. As used herein “ubiquitin conjugating agent” refers to a ubiquitin agent, preferably a protein, capable of transferring or attaching ubiquitin moiety to a ubiquitin ligating agent. In some cases, the ubiquitin conjugating agent is capable of directly transferring or attaching ubiquitin moiety to lysine residues in a target protein (Hershko et al. (1983) J. Biol. Chem. 258:8206-8214). In a preferred embodiment, the ubiquitin conjugating agent is capable of transferring or attaching ubiquitin moiety to a mono- or poly-ubiquitin moiety preferably attached to a ubiquitin agent or target protein. In a preferred embodiment, the ubiquitin conjugating agent is capable of transferring ubiquitin moiety to a mono- or poly-ubiquitinated ubiquitin ligating agent.

In a preferred embodiment the ubiquitin conjugating agent is an E2. In a preferred embodiment, ubiquitin moiety is transferred from E1 to E2. In a preferred embodiment, the transfer results in a thiolester bond formed between E2 and ubiquitin moiety. In a preferred embodiment, E2 is capable of transferring or attaching ubiquitin moiety to an E3, defined below.

In the methods and compositions of the present invention, the ubiquitin activating agent comprises an amino acid sequence or a nucleic acid sequence corresponding to a sequence of an Genbank data base accession number listed in Table 2 below and incorporated herein by reference.

TABLE 2

| | | Accession No. | Accession No. |
|---|---|---|---|
| | | (nucleic acid | (amino acid |
| Name | ALIAS | sequences) | sequences) |
| UBE2D1 Hs UBC4/5 | UBE2D1, UBCH5A, UBC4/5 homolog | NM_003338.1 | NP_003329.1 |
| homolog | | | |
| UBC9 Gallus gallus | UBC9, SUMO-conjugating enzyme | AB069964.1 | BAB68210.1 |
| UBC9 Mus musculus | mUB69 | U76416.1 | AAB18790.1 |
| UBC9/UBE21 Hs ?? | UBE21 | U45328.1 | AAA86662.1 |
| UBC9 isoform/MGC: 3994 | MGC: 3994, IMAGE: 2819732, UBC9 | BC004437.1 | AAH04437.1 |
| Hs | isoform | NM_003345.1 | NP_003336.1 |
| UBC9 Hs | UBC9, UBE21 | | |
| FTS homolog Hs + 1aa | fused toes homolog, FLJ13258 | NM_022476.1 | NP_071921.1 |
| FLJ13988 Hs | FLJ13988, clone Y79AA1002027, sim | AK024050.1 | BAB14800.1 |
| MGC: 13396 Hs | to E2-18 | BC010900.1 | AAH10900.1 |
| UBE2V2 Hs | MGC: 13396, IMAGE: 4081461 | NM_003350.2 | NP_003341.1 |
| MGC: 10481 Hs | UBE2V2, EDAF-1, MMS2, UEV2, | BC004862.1 | AAH04862.1 |
| XM_054332.1 Hs | DDVIT1, ED | XM_054332.1 | XP_054332.1 |
| FLJ13855 Hs | MGC: 10481, IMAGE: 3838157 | XM_030444.3 | XP_030444.1 |
| E2-230K homolog Hs | | NM_022066.1 | NP_071349.1 |
| UBE2V2 Hs | FLJ13855 | NM_003339.1 | NO_003330.1 |
| UBE2D3 Hs 1 SNP | E2-230K ortholog, FLJ12878, | NM_003340.1 | NP_003331.1 |
| Non-canon Ub-conj Enz | KIAA1734 | NM_016336.2 | NP_057420.2 |
| (NCUBE1) | UBE2D2, UBCH5B, UBC4, UBC4/5 | NM_014176.1 | NP_054895.1 |
| HSPC150 Hs | homolog | NM_016252.1 | NP_057336.1 |
| Brain 1AP repeat contain | UBE2D3, UBCH5C, UBC4/5 homolog | | |
| 6 (BIRC6) | NCUBE1, HSU93243, HSPC153, CGI- | | |
| | 76 | | |
| | BIRC6, KIAA1289, apollon | | |
| UBC8 Mus | E2-20K, UBE2H | NM_009459.1 | NP_033485.1 |
| UBC8 Hs | UBE2H, UBCH, UBCH2, UBC8 | NM_003344.1 | NP_003335.1 |
| UBC8 Hs 6SNP | homolog | NM-003344.1 | NP-003335.1 |
| UBC8 Hs no 5′ | UBE2H, UBCH, UBCH2, UBC8 | | |
| | homolog | | |
| RAD6 homolog Hs | UBE2B, RAD6B, HHR6B, UBC2, | NM_003337.1 | NP_003328.1 |
| | RAD6 homolog | | |
| UBE2V1 var 3 Hs | UBE2V1, CIR1, UEV1, UEV1A, | NM_022442.2 | NP_071887.1 |
| UBE2V1 var 1 Hs early | CROC-1, CRO | NM_021988.2 | NP_068823.1 |
| stop, 56aa | UBE2V1, CIR1, UEV1, UEV1A, | NM_003349.3 | NP_003340.1 |
| UBE2V1 var 2 Hs | CROC-1, CRO | | |
| | UBE2V1, CIR1, UEV1, UEV1A, | | |
| | CROC-1, CRO | | |
| UBE2L6 Hs | UBE2L6, UBCH8, RIG-B | NM_004223.1 | NP_004214.1 |
| UBE2L3 Hs 2 SNP | UBE2L3, UBCH7 | NM_003347.1 | NP_003338.1 |
| UBE2E1 Hs | UBE2E1, UBCH6, UBC4/5 homolog | NM_003341.1 | NP_003332.1 |
| RAD6/UBE2A Hs | UBE2A, RAD6A, HHR6A, UBC2, | NM_003336.1 | NP_003327.1 |
| UBE2E3 Hs | RAD6 homolog | NM_006357.1 | NP_006348.1 |
| UBC12/UBE2M Hs | UBE2E3, UBCH9, UBC4/5 homolog | NM_003969.1 | NP_003960.1 |
| UBC7/UBE2G1 Hs | UBE2M, HUBC12, UBC12 homolog | NM_003342.1 | NP_003333.1 |
| | UBE2G1, UBC7 homolog | | |
| Huntingtin interact prot 2 | HIP2, LIG, E2-25K | NM_005339.2 | NP_005330.1 |
| (HIP2) Hs | LIG, HIP2 alternative splicing form | ABO22436.1 | BAA78556.1 |
| LIG/HIP2 variant Hs | | | |
| UBC6p Hs | UBC6p, UBC6 | NM_058167.1 | NP_477515.1 |
| UBC6 Hs | UBC6 | AF296658.1 | AAK52609.1 |
| HBUCE1/UBE2D2 var | HBUCE1, LOC51619 | NM_015983.1 | NP_057067.1 |
| Hs | UBE2G2, UBC7 homolog | XM_036087.1 | XP_036087.1 |
| UBE2G2/UBC7 homolog | NCE2 | NM_080678.1 | NP_542409.1 |
| Hs | CDC34, E2-CDC34, E2-32 | NM_004359.1 | NP_004350.1 |
| NEDD8-conj enzyme 2 | complementing | BC000848.1 | AAH00848.1 |
| (NCE2) Hs | IMAGE: 3458173 | | |
| CDC34 Hs | | | |
| IMAGE: 3458173/NICE-5 | | | |
| var | | | |
| UBE2C Hs | UBE2C, UBCH10 | NM_007019.1 | NP_008950.1 |
| UBE2C possible short | UBE2C, UBCH10 | NM_007019.1 | NP_008950.1 |
| form Hs | | | |
| UBC3/UBE2N Hs | UBE2N, UBCH-BEN, UBC13 hom., | NM_003348.1 | NP_003339.1 |
| FLJ25157 Hs | sim to bend | AK057886.1 | BAB71605.1 |
| TSG101 Hs 1 SNP | FLJ25157, highly similar to E2-23 | NM_006292.1 | NP_006283.1 |
| MGC: 21212/NICE-5 var | Tumor susceptibility gene 101 | BC017708.1 | AAH17708.1 |
| Hs | MCG: 21212, IMAGE: 3907760, sim to | | |
| | NICE-5 | | |

Sequences encoding a ubiquitin conjugating agent may also be used to make variants thereof that are suitable for use in the methods and compositions of the present invention. The ubiquitin conjugatin agents and variants suitable for use in the methods and compositions of the present invention may be made as described herein.

In a preferred embodiment, the E2 used in the methods and compositions of the present invention comprises an amino acid sequence or nucleic acid sequence of a sequence corresponding to an Genbank data base accession number in the following list: AC37534, P49427, CAA82525, AAA58466, AAC41750, P51669, AM91460, AAA91461, CAA63538, AAC50633, P27924, AAB36017, Q16763, AAB86433, AAC26141, CAA04156, BAA11675, Q16781, NP_{—}003333, BAB18652, AAH00468, CAC16955, CAB76865, CAB76864, NP_{—}05536, 000762, XP_{—}009804, XP_{—}009488, XP_{—}006823, XP_{—}006343, XP_{—}005934, XP_{—}002869, XP_{—}003400XP_{—}009365, XP_{—}010361, XP_{—}004699, XP_{—}004019, O14933, P27924, P50550, P52485, P51668, P51669, P49459, P37286, P23567, P56554, and CAB45853, each of which is incorporated herein by reference. Particularly preferred are sequences corresponding to Genbank data base accession numbers NP003331, NP003330, NP003329, P49427, AAB53362, NP008950, XP009488and AAC41750, also incorporated by reference. The skilled artisan will appreciate that many different E2 proteins and isozymes are known in the filed and may be used in the present invention, provided that the E2 has ubiquitin conjugating activity. Also specifically included within the term “E2” are variants of E2, which can be made as described herein.

In a preferred embodiment, the E2 used in the methods and compositions of the present invention comprises an amino acid sequence or nucleic acid sequence of a sequence disclosed in FIGS. 25-34 or as represented by the accession numbers in Table 2. The skilled artisan will appreciate that many different E2 proteins and isozymes are known in the filed and may be used in the present invention, provided that the E2 has ubiquitin conjugating activity. Also specifically included within the term “E2” are variants of E2, which can be made as described herein.**

In some embodiments, E2 has a tag, as defined herein, with the complex being referred to herein as “tag-E2”. Preferred E2 tags include, but are not limited to, labels, partners of binding pairs and substrate binding elements. In a most preferred embodiment, the tag is a His-tag or GST-tag.

In some embodiments, the methods of the present invention comprise the use of a ubiquitin ligating agent. As used herein “ubiquitin ligating agent” refers to a ubiquitin agent, preferably a protein, capable of transferring or attaching a ubiquitin moiety to a target molecule. In some cases, the ubiquitin agent is capable of transferring or attaching ubiquitin moiety to itself or another ubiquitin ligating agent. In a preferred embodiment, the ubiquitin ligating agent is an E3.

As used herein “E3” refers to a ubiquitin ligating agent comprising one or more subunits, preferably polypeptides, associated with the activity of E3 as a ubiquitin ligating agent (i.e., associated with the ligation or attachment of ubiquitin moiety to a target protein, and in some cases, to itself or another E3). In a preferred embodiment, E3 is a member of the HECT domain E3 ligating agents. In another preferred embodiment, E3 is a member of the RING finger domain E3 ligating agents. In a preferred embodiment, E3 comprises a ring finger subunit and a Cullin subunit. Examples of RING finger polypeptides suitable for use in the methods and compositions of the present invention include, but are not limited to, ROC1, ROC2 and APC11. Examples of Cullin polypeptides suitable for use in the methods and compositions of the present invention include, but are not limited to, CUL1, CUL2, CUL3, CUL4A, CUL4B, CUL5 and APC2. In another preferred embodiment, the E3 is mdm2, as shown in FIG. 19.

In the methods and compositions of the present invention, the ubiquitin ligating agent comprises an amino acid sequence or a nucleic acid sequence of a sequence corresponding to an accession number in the Genbank data base, European Molecular Biology Laboratories (EMBL) data base, or ENSEMBL data base (a joint project of the European Molecular Biology Laboratories and the Sanger Institute) listed in Table 3 below and incorporated herein by reference. The accession numbers from the Genbank data base can be found as stated above. The accession numbers from the EMBL data base are found at www.embl-heidelberg.de. The accession numbers from the ENSEMBL data base are found at www.ensembl.or.

TABLE 3

| Accession | Accession | Accession | Accession | Accession | Accession | Accession | Accession | Accession |
|---|---|---|---|---|---|---|---|---|
| No | No. | No. | No. | No. | No | No | No. | No. |
| AAD15547 | AAH22038 | O75485 | Q96BD4 | Q96K03 | Q96T88 | Q9BYV6 | Q9H073 | Q9H920 |
| AAF42995 | AAH22403 | O75592 | Q96BD | Q96K19 | Q99496 | Q9BZX6 | Q9H083 | Q9H9B0 |
| AAF91315 | AAH22510 | O75598 | 5Q96BE6 | Q96K21 | Q99579 | Q9BZX7 | Q9H0A6 | Q9H9B5 |
| AAF97687 | AAL30771 | O75615 | Q96BH1 | Q96KD9 | Q99675 | Q9BZX8 | Q9H0M8 | Q9H9P5 |
| AAG50176 | AAL31641 | O75866 | Q96BL1 | Q96KL0 | Q99942 | Q9BZX9 | Q9H0V6 | Q9H9T2 |
| AAG50180 | AAL36460 | O76050 | Q96BM5 | Q96KM9 | Q9BPW2 | Q9BZY0 | Q9H0X6 | Q9H9V4 |
| AAG53500 | AAL40179 | O76064 | Q96BQ3 | Q96LD4 | Q9BQ47 | Q9BZY1 | Q9H270 | Q9H9Y7 |
| AAG53509 | AAL40180 | O94896 | Q96BS3 | Q96M70 | Q9BQV0 | Q9BZY2 | Q9H2A8 | Q9HA51 |
| AAH00832 | AAL76101 | O94941 | Q96BX2 | Q96MJ7 | Q9BRZ2 | Q9BZY3 | Q9H2S3 | Q9HAC1 |
| AAH02922 | CAC81706 | O94972 | Q96C24 | Q96MT1 | Q9BS04 | Q9BZY4 | Q9H2S4 | Q9HAM2 |
| AAH04978 | CAC85986 | O95159 | Q96CA5 | Q96MX5 | Q9BSE9 | Q9BZY5 | Q9H2S5 | Q9HAP7 |
| AAH05375 | CAD19102 | O95247 | Q96CC2 | Q96MZ7 | Q9BSL8 | Q9BZY6 | Q9H348 | Q9HBD2 |
| AAH13580 | O00237 | O95277 | Q96D24 | Q96NI4 | Q9BSM1 | Q9BZY8 | Q9H463 | Q9HCL8 |
| AAH15738 | O00463 | O95604 | Q96D38 | Q96NS4 | Q9BSV9 | Q9BZY9 | Q9H4C2 | Q9HCR0 |
| AAH16174 | O00635 | O95627 | Q96D59 | Q96NT2 | KIAA066 | Q9C017 | Q9H4C3 | Q9HCR1 |
| AAH16924 | O14616 | O95628 | Q96DB4 | Q96P09 | Q9BTC5 | Q9C018 | Q9H4C4 | Q9HCR2 |
| AAH17370 | O14686 | O96028 | Q96DV2 | Q96PF7 | Q9BTD9 | Q9C019 | Q9H4C5 | Q9HCS6 |
| AAH17585 | O15057 | Q14527 | Q96DV3 | Q96PH3 | Q9BU73 | Q9C021 | Q9H4J2 | Q9NPN4 |
| AAH17592 | O15262 | Q14536 | Q96DX4 | Q96PK3 | Q9BUW4 | Q9C025 | Q9H5E4 | Q9NPP8 |
| AAH17707 | O15344 | Q14848 | Q96DY5 | Q96PM5 | Q9BUZ4 | Q9C026 | Q9H5F1 | Q9NPQ1 |
| AAH18104 | O43164 | Q15156 | Q96EL5 | Q96PR5 | Q9BV68 | Q9C027 | Q9H5K0 | Q9NQ86 |
| AAH18107 | O43255 | Q15290 | Q96EP1 | Q96PU4 | Q9BVG3 | Q9C029 | Q9H5L8 | Q9NQP8 |
| AAH18198 | O43269 | Q15521 | Q96EP8 | Q96PX1 | Q9BW41 | Q9C030 | Q9H5P2 | Q9NR13 |
| AAH18337 | O43270 | Q15959 | Q96EQ8 | Q96QB5 | Q9BW90 | Q9C031 | Q9H5S6 | Q9NRL2 |
| AAH18647 | O43567 | Q16030 | Q96F06 | Q96QB6 | Q9BWF2 | Q9C032 | Q9H647 | Q9NRT4 |
| AAH19283 | O60272 | Q92550 | Q96F37 | Q96QY9 | Q9BWL5 | Q9C033 | Q9H6D9 | Q9NRT6 |
| AAH19355 | O60291 | Q92897 | Q96F67 | Q96RF3 | Q9BWP7 | Q9C034 | Q9H6S6 | Q9NS55 |
| AAH20556 | O60372 | Q969K3 | Q96GF1 | Q96RF8 | Q9BX37 | Q9C035 | Q9H6W8 | Q9NS56 |
| AAH20964 | O60630 | Q969Q1 | Q96GT5 | Q96RW5 | Q9BXI1 | Q9C036 | Q9H6Y7 | Q9NS56 |
| AAH20984 | O75150 | Q969V5 | Q96H69 | Q96SH4 | Q9BY78 | Q9C037 | Q9H748 | Q9NS91 |
| AAH20994 | KIAA0661 | Q96A37 | Q96IB6 | Q96SJ1 | Q9BYE7 | Q9C038 | Q9H874 | Q9NSR1 |
| AAH21258 | O75162 | Q96A61 | Q96ID9 | Q96SL3 | Q9BYV2 | Q9C039 | Q9H890 | Q9NSX7 |
| AAH21570 | O75188 | Q96AK4 | Q96J90 | Q96SR5 | Q9BYV3 | Q9C040 | Q9H8K2 | Q9NTX6 |
| AAH21571 | O75341 | Q96AX9 | Q96JD3 | Q96T06 | Q9BYV4 | Q9C0B0 | Q9H8V9 | Q9NTX7 |
| AAH21925 | O75382 | Q96BD3 | Q96JL5 | Q96T18 | Q9BYV5 | Q9C0G7 | Q9H8W5 | Q9NU68 |
| Q9NUH2 | Q9NZS9 | Q9UIG0 | 9UQPQ7 | O15151 | Q9BXT8 | O94822 | Q13263 | |
| Q9NUR4 | Q9NZT8 | Q9UIG1 | Q9UPR2 | O15541 | Q9BYM8 | O95376 | Q13489 | |
| Q9NUW5 | Q9P0J9 | Q9UJ97 | Q9UQI1 | O60858 | Q9BZR9 | P15918 | Q13490 | |
| Q9NVD5 | Q9P0P0 | Q9UJJ8 | Q9Y225 | O75678 | Q9H000 | P19474 | Q13702 | |
| Q9NVP6 | Q9P115 | Q9UJL3 | Q9Y254 | P14373 | Q9NS80 | P22681 | Q14839 | |
| Q9NW38 | Q9P1Y6 | Q9UJR9 | Q9Y2E6 | P28328 | Q9NV58 | P29590 | Q15326 | |
| Q9NWD2 | Q9P200 | Q9UJV3 | Q9Y2N1 | P35226 | Q9UDY6 | P35227 | Q92785 | |
| Q9NWX1 | Q9P2G1 | Q9UKI6 | Q9Y3C5 | P46100 | Q9UHC7 | P36406 | Q99728 | |
| Q9NX39 | Q9P2L3 | Q9UKV5 | Q9Y3V1 | P51948 | Q9ULX5 | P38398 | Q9HCM9 | |
| Q9NXC0 | Q9P2M3 | Q9ULK6 | Q9Y3V3 | Q12899 | Q9UMT8 | P49754 | Q9NVW2 | |
| Q9NXD0 | Q9UBF6 | Q9ULT6 | Q9Y4I0 | Q12933 | Q9Y4X5 | P50876 | Q9NYG5 | |
| Q9NXI6 | Q9UDN7 | Q9ULW4 | Q9Y4K3 | Q12986 | Q9Y508 | P53804 | Q9ULV8 | |
| Q9NZ15 | Q9UEK4 | Q9UMH1 | Q9Y4L5 | Q13049 | O00623 | P98170 | Q9UPN9 | |
| Q9NZB4 | Q9UF32 | Q9UMQ2 | Q9Y577 | Q13064 | O15164 | Q06587 | Q9Y252 | |
| Q9NZE3 | Q9UHE7 | Q9UNR9 | Q9Y5M7 | Q13114 | O60683 | Q12873 | | |
| Q9NZE9 | Q9UHW2 | Q9UPQ2 | Q9Y6E4 | Q13434 | O75677 | Q13191 | | |
| Q9NZN6 | Q9UID0 | Q9UPQ4 | Q9Y6U1 | Q14258 | O75679 | Q13233 | | |

| | | | | |
|---|---|---|---|---|
| Hect domain proteins | Ringfinger domain proteins | T14346 | BAB23311 | AAL13848 |
| (Embl data base) | (GenBank | NP_008944 | T40821 | XP_004990 |
| AAH19105 | data base) | S66562 | NP_192994 | BAB29387 |
| AAH19345 | AAF50078 | NP_008945 | AAF57824 | BAA92558 |
| AAH21144 | AAH21525 | NP_032421 | NP_080106 | AAG45422 |
| O00307 | AAH02582 | AAK33088 | T37964 | AAF36454 |
| O00308 | NP_055486 | AAL39551 | NP_035798 | AAF36455 |
| O14996 | BAB13352 | NP_175982 | BAB14280 | AAK14420 |
| O15029 | NP_492389 | AAF68076 | XP_084941 | BAA74919 |
| O15033 | XP_048020 | AAF68077 | AAH15380 | BAB24805 |
| O15036 O43165 | BAB28637 | AAH11571 | XP_080159 | BAB30794 |
| O43584 | BAA20780 | XP_052430 | AAF08298 | NP_004229 |
| O94970 | T39585 | AAF68079 | BAA19217 | O08759 |
| O95071 | NP_060239 | AAH04712 | T01491 | AAH19345 |
| O95714 | T39007 | T38951 | CAB92704 | NP_011374 |
| Q15386 | BAA92539 | BAA23711 | CAB09785 | NP_056092 |
| Q15751 | CAC42101 | BAB13451 | NP_177189 | AAH21144 |
| Q96BP4 | XP_083009 | AAF46512 | XP_030186 | NP_056986 |
| Q96CZ2 | AAF79338 | NP_000453 | AAF61856 | B38919 |
| Q96DE7 | NP_060382 | AAL29143 | XP_057408 | T38617 |
| Q96F34 | AAH00621 | AAL27259 | Q9PUN2 | AAH06848 |
| Q96F66 | AAH09271 | AAF36539 | CAB99103 | NP_490834 |
| Q96GR7 | AAC62434 | BAA84697 | NP_195908 | NP_010745 |
| Q96J02 | AAF51314 | NP_499392 | AAH11391 | CAB95249 |
| Q96PU5 | T21546 | AAF68080 | NP_012570 | |
| Q9BUI0 | NP_188346 | I83196 | AAF52899 | |
| Q9BUI6 | AAF49328 | NP_057407 | AAF88143 | |
| Q9BVR2 | XP_082286 | AAF28950 | AAF68614 | |
| Q9BXZ4 | NP_035020 | XP_052223 | BAA20771 | |
| Q9BY75 | NP_501120 | AAF68082 | BAB13419 | |
| Q9H0M0 | NP_055636 | AAF68083 | NP_011051 | |
| Q9H2G0 | NP_003913 | T41750 | AAH13645 | |
| Q9H2W4 | BAB02722 | AAH11658 | Q9CUN6 | |
| Q9H451 | NP_497697 | NP_114087 | XP_046129 | |
| Q9H783 | NP_490865 | Q05086 | A38920 | |
| Q9H9E9 | T14761 | T49744 | AAB47756 | |
| Q9HCC7 | AAC83345 | AAC51324 | Q92462 | |
| Q9HCH9 | S70642 | BAA92571 | NP_113671 | |
| Q9NPL3 | AAG53076 | BAB30733 | CAA57291 | |
| Q9NPS9 | CAA03915 | NP_500283 | XP_087357 | |
| Q9NT88 | XP_085770 | AAK28419 | AAC41731 | |
| Q9NWS4 | CAC09387 | NP_446441 | BAB69424 | |
| Q9NXC0 | NP_055421 | BAA86445 | T37900 | |
| Q9NZS4 | NP_523779 | NP_190877 | T14317 | |
| Q9P0A9 | XP_038999 | Q9HCE7 | P51593 | |
| Q9P2L3 | AAD51453 | AAF50332 | AAH04085 | |
| Q9P2M6 | AAB49301 | AAH09527 | BAA21482 | |
| Q9P2P5 | T49799 | NP_490750 | NP_012915 | |
| Q9UDU3 | AAG16783 | XP_003492 | AAF48495 | |
| Q9UFZ7 | NP_195572 | T37736 | XP_045232 | |
| Q9UII4 | AAH21470 | AAF47474 | AAF50913 | |
| Q9ULT8 Q9Y4D8 | NP_078878 | | T00390 | |
| Q9HAU4 | NP_073576 | | NP_476753 | |
| Q9HCE7 | XP_028151 | | T46412 | |
| P46934 | P46934 | | XP_045095 | |
| Q05086 | BAB28001 | | NP_113584 | |
| Q14669 | NP_004658 | | NP_495842 | |
| Q15034 | P46935 | | AAC04845 | |
| | NP_524296 | | XP_030175 | |
| | | | 1C4Z | |

| | | | |
|---|---|---|---|
| Ringfinger domain proteins | ENSP00000282135 | ENSP00000255977 | ENSP00000265742 |
| (Ensembl data base) | ENSP00000280460 | ENSP00000283490 | ENSP00000269475 |
| ENSP00000259945 | ENSP00000280461 | ENSP00000262370 | ENSP00000265290 |
| ENSP00000254436 | ENSP00000217740 | ENSP00000253024 | EN3P00000222597 |
| ENSP00000066988 | ENSP00000227588 | ENSP00000282369 | ENSP00000292307 |
| ENSP00000275736 | ENSP00000259944 | ENSP00000253571 | ENSP00000265267 |
| ENSP00000275735 | ENSP00000279757 | ENSP00000288913 | ENSP00000263220 |
| ENSP00000203439 | ENSP00000274773 | ENSP00000288918 | ENSP00000216225 |
| ENSP00000013772 | ENSP00000276311 | ENSP00000276573 | ENSP00000293538 |
| ENSP00000225283 | ENSP00000166144 | ENSP00000237308 | EN6900000229766 |
| ENSP00000246907 | ENSP00000292363 | ENSP00000238203 | ENSP00000242239 |
| ENSP00000225285 | ENSP00000264616 | ENSP00000227451 | ENSP00000274616 |
| ENSP00000225286 | ENSP00000272390 | ENSP00000244360 | ENSP00000286773 |
| ENSP00000230239 | ENSP00000272396 | ENSP00000244359 | ENSP00000273480 |
| ENSP00000286909 | ENSP00000264767 | ENSP00000281105 | ENSP00000217173 |
| ENSP00000286910 | ENSP00000255499 | ENSP00000268907 | ENSP00000290337 |
| ENSP00000280609 | ENSP00000264614 | ENSP00000292962 | ENSP00000281930 |
| ENSP00000263651 | ENSP00000262482 | ENSP00000280804 | ENSP00000257575 |
| ENSP00000261395 | ENSP00000261481 | ENSP00000287546 | ENSP00000287212 |
| ENSP00000277584 | ENSP00000261658 | ENSP00000248980 | ENSP00000290788 |
| ENSP00000224833 | ENSP00000288774 | ENSP00000287559 | ENSP00000282455 |
| ENSP00000254604 | ENSP00000261675 | ENSP00000264926 | ENSP00000254247 |
| ENSP00000240395 | ENSP00000266880 | ENSP00000261737 | ENSP00000290649 |
| ENSP00000240318 | ENSP00000243674 | ENSP00000170447 | ENSP00000274542 |
| ENSP00000286945 | ENSP00000284638 | ENSP00000270944 | ENSP00000224944 |
| ENSP00000281874 | ENSP00000247668 | ENSP00000289726 | ENSP00000281418 |
| EN9P00000240802 | ENSP00000285317 | ENSP00000230099 | ENSP00000289883 |
| ENSP00000267825 | ENSP00000278480 | ENSP00000237455 | ENSP00000255325 |
| ENSP00000254586 | ENSP00000240159 | ENSP00000263550 | ENSP00000255326 |
| ENSP00000293123 | ENSP00000294256 | ENSP00000264198 | ENSP00000292543 |
| ENSP00000285805 | ENSP00000279766 | ENSP00000263464 | ENSP00000277534 |
| ENSP00000257633 | ENSP00000288204 | ENSP00000259604 | ENSP00000260947 |
| ENSP00000266119 | ENSP00000269439 | ENSP00000265673 | ENSP00000278455 |
| ENSP00000233630 | ENSP00000268061 | ENSP00000248983 | ENSP00000278454 |
| ENSP00000264033 | ENSP00000268058 | ENSP00000269391 | ENSP00000274694 |
| ENSP00000275619 | ENSP00000268059 | ENSP00000249007 | ENSP00000217740 |
| ENSP00000275637 | ENSP00000268060 | ENSP00000242719 | ENSP00000262952 |
| ENSP00000280063 | ENSP00000261825 | ENSP00000217169 | ENSP00000268154 |
| ENSP00000276333 | ENSP00000288587 | ENSP00000253642 | ENSP00000265756 |
| ENSP00000263651 | ENSP00000275693 | ENSP00000227758 | ENSP00000277490 |
| ENSP00000278302 | ENSP00000244061 | ENSP00000291190 | ENSP00000266625 |
| ENSP00000264122 | ENSP00000272598 | ENSP00000261537 | ENSP00000266624 |
| ENSP00000284559 | ENSP00000289818 | ENSP00000291733 | ENSP00000258147 |
| ENSP00000266252 | ENSP00000238349 | ENSP00000274782 | ENSP00000258148 |
| ENSP00000278350 | ENSP00000280266 | ENSP00000271287 | ENSP00000258149 |
| ENSP00000259847 | ENSP00000242855 | ENSP00000261445 | ENSP00000264512 |
| ENSP00000274855 | ENSP00000276688 | ENSP00000245836 | ENSP00000261212 |
| ENSP00000259930 | ENSP00000280268 | ENSP00000267291 | ENSP00000262642 |
| ENSP00000217214 | ENSP00000274811 | ENSP00000292195 | ENSP00000264359 |
| ENSP00000283330 | ENSP00000268363 | ENSP00000216420 | ENSP00000217537 |
| ENSP00000263535 | ENSP00000274828 | ENSP00000261464 | ENSP00000264777 |
| ENSP00000291416 | ENSP00000235150 | ENSP00000260076 | ENSP00000287880 |
| ENSP00000291414 | ENSP00000211960 | ENSP00000284244 | ENSP00000272674 |
| ENSP00000253769 | ENSP00000262843 | ENSP00000292545 | ENSP00000272662 |
| ENSP00000274786 | ENSP00000266952 | ENSP00000242669 | ENSP00000293245 |
| ENSP00000289896 | ENSP00000288300 | ENSP00000288848 | ENSP00000283875 |
| ENSP00000289898 | ENSP00000291134 | ENSP00000261809 | ENSP00000262642 |
| ENSP00000265771 | ENSP00000261947 | ENSP00000262952 | ENSP00000259865 |
| ENSP00000229866 | ENSP00000288715 | ENSP00000245937 | ENSP00000217908 |
| ENSP00000286475 | ENSP00000222704 | ENSP00000275970 | ENSP00000255004 |
| ENSP00000256257 | ENSP00000293938 | ENSP00000238647 | ENSP00000275184 |
| ENSP00000253554 | ENSP00000266030 | ENSP00000268850 | ENSP00000275183 |
| ENSP00000259654 | ENSP00000287335 | ENSP00000291963 | ENSP00000200457 |
| ENSP00000280266 | ENSP00000256649 | ENSP00000286349 | ENSP00000261537 |
| ENSP00000259941 | ENSP00000249240 | ENSP00000257600 | ENSP00000257100 |
| ENSP00000259940 | ENSP00000253953 | ENSP00000281843 | ENSP00000286349 |
| ENSP00000270086 | ENSP00000267073 | ENSP00000261245 | ENSP00000252445 |
| ENSP00000289140 | ENSP00000271813 | ENSP00000245888 | ENSP00000294213 |
| ENSP00000225507 | ENSP00000248492 | ENSP00000222704 | ENSP00000259939 |
| ENSP00000261593 | ENSP00000265981 | ENSP00000245419 | ENSP00000236892 |
| ENSP00000257847 | ENSP00000270280 | ENSP00000272023 | ENSP00000238001 |
| ENSP00000262881 | ENSP00000270279 | ENSP00000274068 | ENSP00000274657 |
| ENSP00000222033 | ENSP00000254959 | ENSP00000275233 | ENSP00000274799 |
| ENSP00000290048 | | | |
| ENSP00000274327 | | | |

Sequences encoding a ubiquitin activating agent may also be used to make variants thereof that are suitable for use in the methods and compositions of the present invention. The ubiquitin ligating agents and variants suitable for use in the methods and compositions of the present invention may be made as described herein.

In a preferred embodiment, RING finger subunits include, but are not limited to, polypeptides having an amino acid sequence corresponding to Genbank accession numbers AAD30147, AAD30146, or 6320196, incorporated herein by reference.

In a preferred embodiment, Cullins include, but are not limited to, polypeptides having an amino acid sequence corresponding to Genbank accession number 4503161, AAC50544, AAC36681, 4503163, AAC51190, AAD23581, 4503165, AAC36304, AAC36682, AAD45191, AAC50548, Q13620,4503167, or AAF05751, each of which is incorporated herein by reference. In addition, in the context of the invention, each of the RING finger proteins and Cullins encompass variants of the known or listed sequences, as described herein.

These E3 ligating agents and variants may be made as described herein. In a preferred embodiment, nucleic acids used to make the RING finger proteins include, but are not limited to, those having the nucleic acid sequences disclosed in Genbank accession numbers AF142059, AF142060 and nucleic acids 433493 to 433990 of NC 001136. In a preferred embodiment, Cullins are made from nucleic acids including, but not limited to, those having nucleic acid sequences disclosed in Genbank accession numbers NM 003592, U58087, AF062536, AF126404, NM 003591, U83410, NM 003590, AB014517, AF062537, AF064087, AF077188, U58091, NM 003478, X81882 and AF191337, each of which is incorporated herein by reference. As described herein, variants of these sequences are also encompassed by the invention.

In a preferred embodiment, E3 comprises the RING finger protein/Cullin combination APC11/APC2. In another preferred embodiment, E3 comprises the RING finger protein/Cullin combination ROC1/CUL1. In yet preferred embodiment, E3 comprises the RING finger protein/Cullin combination ROC1/CUL2. In still another preferred embodiment, E3 comprises the RING finger protein/Cullin combination ROC2/CUL5. However, the skilled artisan will appreciate that any combination of E3 components may be produced and used in the invention described herein.

In an alternate embodiment, E3 comprises the ligase E3-alpha, E3A (E6-AP), HERC2, SMURF1, TRAF6, Mdm2, Cbl, Sina/Siah, Itchy, IAP or NEDD-4. In this embodiment, the ligase has the amino acid sequence of that disclosed in Genbank accession number AAC39845, Q05086, CAA66655, CAA66654, CAA66656, AAD08657, NP_{—}002383, XP_{—}006284, AAC51970, XP_{—}013050, BAB39389, Q00987, AAF08298 or P46934, each of which is incorporated herein by reference. As above, variants are also encompassed by the invention. Nucleic acids for making E3 for this embodiment include, but are not limited to, those having the sequences disclosed in Genbank accession numbers AF061556, XM006284, U76247, XM013050, X898032, X98031, X98033, AF071172, Z12020, AB056663, AF199364 and D42055 and variants thereof.

E3 may also comprise other components, such as SKP1 and F-box proteins. The amino acid and nucleic acid sequences for SKP1 correspond to GENBANK accession numbers AAC50241 and U33760, respectively. Many F-box proteins are known in the art and their amino acid and nucleic acid sequences are readily obtained by the skilled artisan from various published sources.

In a preferred embodiment, the E3 components are produced recombinantly, as described herein. In a preferred embodiment, the E3 components are co-expressed in the same host cell. Co-expression may be achieved by transforming the cell with a vector comprising nucleic acids encoding two or more of the E3 components, or by transforming the host cell with separate vectors, each comprising a single component of the desired E3 protein complex. In a preferred embodiment, the RING finger protein and Cullin are expressed in a single host transfected with two vectors, each comprising nucleic acid encoding one or the other polypeptide, as described in further detail in the Examples.

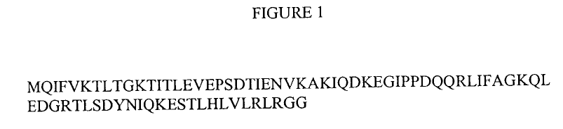

By “ubiquitin moiety” herein is meant a polypeptide which is transferred or attached to another polypeptide by a ubiquitin agent. Ubiquitin moiety includes both ubiquitin and ubiquitin-like molecules. The ubiquitin moiety can comprise a ubiquitin from any species of organism, preferably a eukaryotic species. In preferred embodiments the ubiquitin moiety comprises is a mammalian ubiquitin, and more preferably a human ubiquitin. In a preferred embodiment, the ubiquitin moiety comprises a 76 amino acid human ubiquitin. In a preferred embodiment, the ubiquitin moiety comprises the amino acid set forth in FIG. 1. In other preferred embodiments, the ubiquitin moiety comprises ubiquitinlike molecules having an amino acid sequence or nucleic acid sequence of a sequence corresponding to one of the GENBANK accession numbers disclosed in TABLE 4. Other embodiments utilize variants of ubiquitin, as further described below.

TABLE 4
Ubls

| | | ACCESSION | ACCESSION |
|---|---|---|---|
| | | NUMBERS | NUMBERSS |
| | | (nucleic acid | (nucleic acid |
| Ubl | Alias | sequences) | sequences) |
| Ubiquitin | | NM_002954.2 | NP_002945 |
| NEDD-8 | | NM_006156.1 | NP_006147 |
| ISG-15 | UCRP | NM_005101.1 | NP_005092.1 |
| APG12 | APG12L, MAP1_LC3 | NM_004707.1 | NP_004698.1 |
| APG8 | MAP1_LC3, MAP1A, | NM_022818.2 | NP_073729.1 |
| | 1BLC3 | | |
| Fat10 | Diubiquitin | NM_006398.1 | NP_006389.1 |
| Fau, | FBR-MuSV-associated | NM_001997.2 | NP_001988.1 |
| Fubi | ubiquitously expressed | | |
| | gene, ubiquitin-like protein | | |
| | fubi, 40S ribosomal | | |
| | protein S30, FAU- | | |
| | encoded ubiquitin- | | |
| | like protein | | |
| SUMO-1 | Sentrin1, SMT3C, GMP1, | NM_003352.2 | NP_003343.1 |
| | PIC, SM, SMT3H3 | | |
| SUMO-2 | Sentrin3, SMT3A, | NM_006936.1 | NP_008867.1 |
| | SMT3H1 | | |
| SUMO-3 | SMT3B, SMT3H2, | NM_006937.2 | NP_008868.2 |
| | HSMT3 | | |

As used herein, “poly-ubiquitin moiety” refers to a chain of ubiquitin moieties comprising more than one ubiquitin moiety. As used herein, “mono-ubiquitin moiety” refers to a single ubiquitin moiety. In the methods of the present invention, a mono- or poly-ubiquitin moiety can serve as a substrate molecule for the transfer or attachment of ubiquitin moiety (which can itself be a mono- or polyubiquitin moiety).

In a preferred embodiment, when ubiquitin moiety is attached to a target protein, that protein is targeted for degradation by the 26S proteasome.

As used herein, “ubiquitin moiety” encompasses naturally occurring alleles and man-made variants of ubiquitin or ubiquitin-like molecules. In a preferred embodiment the ubiquitin moiety includes a 76 amino acid polypeptide as described above or variants thereof. In a preferred embodiment, the ubiquitin moiety comprises an amino acid sequence or nucleic acid sequence corresponding to a sequence of GENBANK accession number P02248, incorporated herein by reference.

GENBANK accession numbers and their corresponding amino acid sequences or nucleic acid sequences are found in the Genbank data base. Sequences corresponding to GenBank accession numbers cited herein are incorporated herein by reference. GenBank is known in the art, see, e.g., Benson, D A, et al., Nucleic Acids Research 26:1-7 (1998) and http://www.ncbi.nlm.nih.gov/. Preferably, the ubiquitin moiety has the amino acid sequence depicted in FIG. 1. In a preferred embodiment, variants of ubiquitin moiety have an overall amino acid sequence identity of preferably greater than about 75%, more preferably greater than about 80%, even more preferably greater than about 85% and most preferably greater than 90% of the amino acid sequence depicted in **figure 15A. In some embodiments the sequence identity will be as high as about 93 to 95 or 98%.

In another preferred embodiment, a ubiquitin moiety protein has an overall sequence similarity with the amino acid sequence depicted in FIG. 1 of greater than about 80%, more preferably greater than about 85%, even more preferably greater than about 90% and most preferably greater than 93%. In some embodiments the sequence identity will be as high as about 95 to 98 or 99%.

As is known in the art, a number of different programs can be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl/edu/blast/ README.html]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25:3389-3402. Gapped BLAST uses BLOSUM-62 substitution scores; threshold T parameter set to 9; the two-hit method to trigger ungapped extensions; charges gap lengths of k a cost of 10+k; Xu set to 16, and Xg set to 40 for database search stage and to 67 for the output stage of the algorithms. Gapped alignments are triggered by a score corresponding to ˜22 bits.

A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment may include the introduction of gaps in the sequences to be aligned. In addition, for sequences which contain either more or fewer amino acids than the amino acid sequence depicted in FIG. 1, it is understood that in one embodiment, the percentage of sequence identity will be determined based on the number of identical amino acids in relation to the total number of amino acids. Thus, for example, sequence identity of sequences shorter than that of the sequence depicted in FIG. 1, as discussed below, will be determined using the number of amino acids in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

Ubiquitin moieties of the present invention are polypeptides that may be shorter or longer than the amino acid sequence depicted in FIG. 1. Thus, in a preferred embodiment, included within the definition of ubiquitin moiety are portions or fragments of the amino acid sequence depicted in FIG. 1. In one embodiment herein, fragments of ubiquitin moiety are considered ubiquitin moieties if they are attached to another polypeptide by a ubiquitin agent.

In addition, as is more fully outlined below, ubiquitin moieties of the present invention are polypeptides that can be made longer than the amino acid sequence depicted in FIG. 1; for example, by the addition of tags, the addition of other fusion sequences, or the elucidation of additional coding and non-coding sequences. As described below, the fusion of a ubiquitin moiety to a fluorescent peptide, such as Green Fluorescent Peptide (GFP), is particularly preferred.

In one embodiment, the ubiquitin moiety is an endogenous molecule. That is the ubiquitin moiety is naturally expressed in the cell to be assayed. However, in an alternative embodiment, the ubiquitin moiety, as well as other proteins of the present invention, are exogenous. That is, they are recombinant proteins. A “recombinant protein” is a protein made using recombinant techniques, i.e. through the expression of a recombinant nucleic acid as described below. In a preferred embodiment, the ubiquitin moiety of the invention is made through the expression of a nucleic acid sequence corresponding to GENBANK accession number M26880 or AB003730, or a fragment thereof. In a most preferred embodiment, the nucleic acid encodes the amino acid sequence depicted in FIG. 1.

Accordingly, in a preferred embodiment, the cells may further comprise recombinant nucleic acid that encodes a target protein. The terms “polypeptide” and “protein” may be used interchangeably throughout this application and mean at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline.

The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradation. However, in a preferred embodiment, naturally occurring amino acids are used and the protein is a cellular protein that is either endogenous or expressed recombinantly.

A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes, but is not limited to, the production of a protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below. In a preferred embodiment, the protein is a dominant negative as described herein.

By “target protein” or “substrate protein” or “ubiquitin ligase substrate” herein is meant a protein other than a ubiquitin moiety to which a ubiquitin moiety is bound or attached through the activity of a ubiquitin agent or by the process of ubiquitination. In preferred embodiments, the target protein is a mammalian target protein, and more preferably a human target protein. That is, as used herein, “substrate molecule” or “target substrate” and grammatical equivalents thereof means a molecule, preferably a protein, to which a ubiquitin moiety is bound or attached through the activity of a ubiquitin agent or by the process of ubiquitination. As used herein with reference to the activity of ubiquitin agents, “attachment” refers to the transfer, binding, ligation, and/or ubiquitination of a mono- or polyubiquitin ubiquitin moiety to a substrate molecule. Thus, “ubiquitination” and grammatical equivalents thereof means the attachment, or transfer, binding, and/or ligation of ubiquitin moiety to a substrate molecule; and “ubiquitination reaction” and grammatical equivlents thereof refer to the combining of components under conditions that permit ubiquitination (i.e., the attachment or transfer, binding, and/or ligation of ubiquitin moiety to a substrate molecule).

Also included within the definition of the proteins used in the invention are variant or mutant proteins. In a preferred embodiment, the variant ubiquitin agents are dominant negative mutants or variants. By “dominant negative is meant that the mutant prevents, inhibits or blocks the activity of the wild type molecule. Dominant negative mutants may take many forms. They may be truncations, deletions, or even point mutations. Generally, the variant is modified such that the molecule loses it is activity. Without being bound by theory, it is thought that expression of this mutant inhibits the activity of the wild type molecule, or inhibits signal transduction by molecules in the pathway of the wild type molecule. In addition, dominant negatives bind with, but do not activate their binding partner. That is, the dominant negative can bind to the wild-type binding partner and prevent its activation. In some embodiements, when homo-oligomerization is required for activation, the dominant negative binds with its wild-type counterpart to prevent activation.

In one embodiment, the present invention provides compositions containing protein variants, for example ubiquitin moiety, E1, E2 and/or E3 variants. These variants fall into one or more of three classes: substitutional, insertional or deletional variants. These variants ordinarily are prepared by site specific mutagenesis of nucleotides in the DNA encoding a protein of the present compositions, using cassette or PCR mutagenesis or other techniques well known in the art, to produce DNA encoding the variant, and thereafter expressing the DNA in recombinant cell culture as outlined above. However, variant protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed variants screened for the optimal desired activity. Techniques for making substitution mutations at predetermined sites in DNA having a known sequence are well known, for example, M13 primer mutagenesis and PCR mutagenesis. Rapid production of many variants may be done using techniques such as the method of gene shuffling, whereby fragments of similar variants of a nucleotide sequence are allowed to recombine to produce new variant combinations. Examples of such techniques are found in U.S. Pat. Nos. 5,605,703; 5,811,238; 5,873,458; 5,830,696; 5,939,250; 5,763,239; 5,965,408; and 5,945,325, each of which is incorporated by reference herein in its entirety. Screening of the mutants is performed using the activity assays of the present invention.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the protein are desired, substitutions of an original residue are generally made in accordance with exemplary substitutions listed below.

Original Residue Exemplary Substitutions

| | |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in the above list. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

In one embodiment, the variants typically exhibit the same qualitative biological activity and will elicit the same immune response as the naturally-occurring analogue, although variants also are selected to modify the characteristics of the proteins as needed. Alternatively, the variant may be designed such that the biological activity of the protein is altered. For example, glycosylation sites may be altered or removed.

In an alternative embodiment, the variants modify the transcript of the endogenous wild type molecule rather than the protein or translation product. That is, in this embodiment, the variants are antisense molecules or siRNA molecules. In this embodiment, the transcription product of the ubiquitin agent variant, reduces expression of the wild type protein. Without being bound by theory, it is thought that the antisense molecule or si RNA molecules prevent expression of the wild type molecule.

In a preferred embodiment the variant is an siRNA that targets a ubiquitin agent. When designing siRNA, preferred methods of selecting a target or designing the nucleic acid include: 1. begin with the AUG start codon of the mRNA to be targeted, skip the first 75 bases and scan downstream for AA dinucleotide sequences. Record the occurrence of each AA and the 3′ adjacent 19 nucleotides as potential siRNA target sites. Tuschl, et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex; 2.Check each potential target site and make sure its GC content is between 30-70% and it does not have a stretch of more than 4 Gs or Cs; 3. Check each potential target sites (using BLAST search for human genes) and make sure it does not sit on an intron/exon boundary; 4.Ensure that each potential target site does not contain a SNP; 5.Compare the potential target sites to the appropriate database and eliminate from consideration any target sequences with significant homology to other coding sequences; 6.Select 3 to 4 target sequences along the length of the gene to evaluate whether the 5′, 3′, or medial portions of mRNAs are more susceptible to siRNA induced degradation.

In one embodiment, covalent modifications of polypeptides are included within the scope of this invention. Such covalent modifications generally find use in in vitro assays as described in more detail in U.S. Ser. No. 09/800,770, filed Mar. 6, 2001, which is expressly incorporated herein by reference.

Polypeptides of the present invention may also be modified in a way to form chimeric molecules comprising a first polypeptide fused to another, heterologous polypeptide or amino acid sequence. In one embodiment, such a chimeric molecule comprises a fusion of a substrate molecule (e.g., a ubiquitin moiety, ubiquitin agent, or target protein) with a tag polypeptide which provides an epitope to which an anti-tag antibody can selectively bind. The epitope tag is generally placed at the amino-or carboxyl-terminus of the polypeptide. The presence of such epitope-tagged forms of a polypeptide can be detected using an antibody against the tag polypeptide. Also, providing an epitope tag enables the polypeptide to be readily purified by affinity purification using an anti-tag antibody or another type of affinity matrix that binds to the epitope tag. In an alternative embodiment, the chimeric molecule may comprise a fusion of a polypeptide disclosed herein with an immunoglobulin or a particular region of an immunoglobulin. For a bivalent form of the chimeric molecule, such a fusion could be to the Fc region of an IgG molecule. Tags for components of the invention are defined and described in detail below.

In a preferred embodiment, one or more components of the present invention comprise a tag. By “tag” is meant an attached molecule or molecules useful for the identification or isolation of the attached molecule(s), which are preferably substrate molecules. For example, a tag can be an attachment tag or a label tag. Components having a tag are referred to as “tag-X”, wherein X is the component. For example, a ubiquitin moiety comprising a tag is referred to herein as “tag-ubiquitin moiety”. Preferably, the tag is covalently bound to the attached component. When more than one component of a combination has a tag, the tags will be numbered for identification, for example “tag1-ubiquitin moiety”. Components may comprise more than one tag, in which case each tag will be numbered, for example “tag 1,2-ubiquitin moiety”. Preferred tags include, but are not limited to, a label, a partner of a binding pair, and a surface substrate binding molecule (or attachment tag). As will be evident to the skilled artisan, many molecules may find use as more than one type of tag, depending upon how the tag is used. In a preferred embodiment, the tag or label as described below is incorporated into the polypeptide as a fusion protein. Tags and labels are described in more detail in 68613**, which is incorporated herein by reference.

By “label” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is performed will depend on the label. Preferred labels include, but are not limited to, fluorescent labels (e.g. GFP) and label enzymes.

By “fluorescent label” is meant any molecule that may be detected via its inherent fluorescent properties. Suitable fluorescent labels include, but are not limited to, green fluorescent protein (GFP; Chalfie, et al., Science 263(5148):802-805 (Feb 11,1994); and EGFP; Clontech—Genbank Accession Number U55762 ), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif 94303), luciferase (Ichiki, et al., J. Immunol. 150(12):5408-5417 (1993)), -galactosidase (Nolan, et al., Proc Natl Acad Sci USA 85(8):2603-2607 (April 1988)) and Renilla WO 92/15673; WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558), and Ptilosarcus green fluorescent proteins (pGFP) (see WO 99/49019). All of the above-cited references are expressly incorporated herein by reference.

The production of tag-polypeptides by recombinant means when the tag is also a polypeptide is described below. Production of FLAG-labeled proteins is well known in the art and kits for such production are commercially available (for example, from Kodak and Sigma). Methods for the production and use of FLAG-labeled proteins are found, for example, in Winston et al., Genes and Devel. 13:270-283 (1999), incorporated herein in its entirety, as well as product handbooks provided with the above-mentioned kits.

Production of proteins having His-tags by recombinant means is well known, and kits for producing such proteins are commercially available. Such a kit and its use is described in the QlAexpress Handbook from Qiagen by Joanne Crowe et al., hereby expressly incorporated by reference.

In a preferred embodiment, ubiquitin moiety is in the form of tag-ubiquitin moiety, wherein, tag is a partner of a binding pair. Preferably in this embodiment the tag is FLAG and the binding partner is anti-FLAG. Preferably in this embodiment, a label is attached to the FLAG by indirect labeling. Preferably, the label is a label enzyme. Most preferably, the label enzyme is horseradish peroxidase, which is reacted with a fluorescent label enzyme substrate. Preferably, the label enzyme substrate is Luminol. Alternatively, the label is a fluorescent label.

Another type of covalent modification of a polypeptide included within the scope of this invention comprises altering the native glycosylation pattern of the polypeptide. “Altering the native glycosylation pattern” is intended for purposes herein to mean deleting one or more carbohydrate moieties found in native sequence polypeptide, and/or adding one or more glycosylation sites that are not present in the native sequence polypeptide.

Addition of glycosylation sites to polypeptides may be accomplished by altering the amino acid sequence thereof. The alteration may be made, for example, by the addition of, or substitution by, one or more serine or threonine residues to the native sequence polypeptide (for O-linked glycosylation sites). The amino acid sequence may optionally be altered through changes at the DNA level, particularly by mutating the DNA encoding the polypeptide at preselected bases such that codons are generated that will translate into the desired amino acids.

In a preferred embodiment, the dominant negative is created using cDNA fragments. As used herein, the term “cDNA” means DNA that corresponds to or is complementary to at least a portion of messenger RNA (mRNA) sequence and is generally synthesized from an mRNA preparation using reverse transcriptase or other methods. cDNA as used herein includes full length cDNA, corresponding to or complementary in sequence to full length mRNA sequences, partial cDNA, corresponding to or complementary in sequence to portions of mRNA sequences, and cDNA fragments, also corresponding to or complementary to portions of mRNA sequences. It should be understood that references to a particular “number” of cDNAs or other nucleic acids actually refers to the number of clones, cDNA sequences or species, rather than the number of physical copies of substantially identical sequences present. Moreover, the term is often used to refer to cDNA sequences incorporated into a plasmid or viral vector which can, in turn, be present in a bacterial cell, mammalian packaging cell line, or host cell.

By “CDNA fragment” is meant a portion of a cDNA that is derived by fragmentation of a larger cDNA. cDNA fragments may be derived from partial or full length cDNAs. As will be appreciated, a number of methods may be used to generate cDNA fragments. For example, cDNA may be subjected to shearing forces in solution that can break the covalent bonds of the backbone of the cDNA. In a preferred embodiment, cDNA fragments are generated by digesting cDNA with restriction endonuclease(s). Other methods are well known in the art.

“Partial cDNA” refers to cDNA that comprises part of the nucleic acid sequence which corresponds to or is complementary to the open reading frame (ORF) of the corresponding mRNA.

“Full length cDNA” refers to cDNA that comprises the complete sequence which is complementary to or corresponds to the ORF of the corresponding mRNA. In some instances, which are clear, full length cDNA refers to cDNA that comprises sequence complementary to or corresponding to the 5′ untranslated region (UTR) of the corresponding mRNA, in addition to sequence which is complementary to or corresponds to the complete ORF.

A corresponding mRNA comprises the nucleotide sequence of the mRNA used as template for synthesis of a particular cDNA, or is the template mRNA used for synthesis of a particular cDNA.

The occurrence of alternatively spliced mRNAs in an mRNA pool used to make cDNA may lead to the synthesis of a cDNA which has sequence corresponding to more than one mRNA type. In addition, the cDNA may comprise a nucleotide sequence that is identical to only a segment of an alternatively spliced mRNA.

In a preferred embodiment, libraries comprising expression vectors with random cDNA in sense orientation are provided. In another embodiment, libraries comprising expression vectors with random cDNA in antisense orientation are provided. In another embodiment, libraries comprising a mixture of expression vectors with random cDNAs in sense orientation and antisense orientation are provided. cDNA constructs are described in more detail in U.S. Ser. Nos. 10/142,648, filed May 8, 2002 and U.S. Ser. No. 10/142,662, filed May 8, 2002, both of which are expressly incorporated herein by reference.

Ubiquitin moieties, ubiquitin agents, and target molecules suitable for use in the methods and compositions of the present invention can be cloned and expressed as described below. Thus, probe or degenerate polymerase chain reaction (PCR) primer sequences may be used to find other related or variant ubiquitin moieties, ubiquitin agents, and target proteins from humans or other organisms. As will be appreciated by those in the art, particularly useful probe and/or PCR primer sequences include the unique areas of a nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art. It is therefore also understood that provided along with the sequences in the sequences cited herein are portions of those sequences, wherein unique portions of 15 nucleotides or more are particularly preferred. The skilled artisan can routinely synthesize or cut a nucleotide sequence to the desired length.

Once isolated from its natural source, e.g., contained within a plasmid or other vector or excised therefrom as a linear nucleic acid segment, the recombinant nucleic acid can be further-used as a probe to identify and isolate other nucleic acids. It can also be used as a “precursor” nucleic acid to make modified or variant nucleic acids and proteins.

In a preferred embodiment, the nucleic acids of the invention are part of an expression vector. Using the nucleic acids of the present invention which encode a protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. As another example, operably linked refers to DNA sequences linked so as to be contiguous, and, in the case of a secretory leader, contiguous and in reading fram. However, enhancers do not have to be contiguous. Linking is accomplished by ligation at convenient restriction sites. If such sites do not exist, the synthetic oligonucleotide adapters or linkers are used in accordance with conventional practice. The transcriptional and translational regulatory nucleic acid will generally be appropriate to the host cell used to express the protein; for example, transcriptional and translational regulatory nucleic acid sequences from Bacillus are preferably used to express the protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

A preferred expression vector system is a retroviral vector system such as is generally described in PCT/US97/01019 and PCT/US97/01048, both of which are hereby expressly incorporated by reference. Constructs also are described in U.S. Ser. No. 08/789,333, filed Jan. 23, 1997, and issued Nov. 28, 2000 as U.S. Pat. No. 6,153,380, which is expressly incorporated herein by reference.

Proteins of the present invention are produced by culturing a host cell transformed with an expression vector containing nucleic acid encoding the protein, under the appropriate conditions to induce or cause expression of the protein. The conditions appropriate for protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction.

Appropriate host cells include yeast, bacteria, archaebacteria, fungi, and insect and animal cells, including mammalian cells. Of particular interest are Drosophila melanogaster cells, Pichia pastoris and P. methanolica, Saccharomyces cerevisiae and other yeasts, E. coli, Bacillus subtilis, SF9 cells, SF21 cells, C129 cells, Saos-2 cells, Hi-5 cells, 293 cells, Neurospora, BHK, CHO, COS, and HeLa cells. Of greatest interest are A549, HeLa, HUVEC, Jurkat, BJAB, CHMC, and .

In a preferred embodiment, the proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for a protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase 11 to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

A suitable bacterial promoter is any nucleic acid sequence capable of binding bacterial RNA polymerase and initiating the downstream (3′) transcription of the coding sequence of a protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

Methods for expression and purification of proteins in yeast, bacteria and other cell lines are described in more detail in U.S. Ser. No. 09/800,770, filed Mar. 6, 2001, which is expressly incorporated herein by reference.

The protein may also be made as a fusion protein, using techniques well known in the art. Thus, for example, the protein may be made fusion nucleic acid encoding the peptide or may be linked to other nucleic acid for expression purposes. Similarly, proteins of the invention can be linked to protein labels, such as green fluorescent protein (GFP), red fluorescent protein (RFP), blue fluorescent protein (BFP), yellow fluorescent protein (YFP), etc. The fusions may include other constructs as well, including separation sites such as 2a site and internal ribosomal entry sites IRES, which are particularly useful in the construct as IRES-label to provide a method of tracking infected cells.

Once made, the nucleic acids and/or vectors of the invention find use in a variety of applications, including a variety of screening methods. In a preferred embodiment, the methods comprising introducing a library of nucleic acids and/or vectors into a population or library of cells and screening the library of cells for an altered phenotype as compared to control cells.

By “altered phenotype” herein is meant a detectable change in a phenotype of a cell as compared with control cells, e.g. cells not expressing a variant ubiquitin agent.

Accordingly, the present invention provides methods and compositions comprising expressing different combinations of ubiquitin agents, with ubiquitin moiety that is exogenous or endogenous to the cell, and assaying cell cultures in a variety of functional assays In preferred embodiments, a variant ubiquitin agent such as a dominant negative ubiquitin agent is included in the assay.

Accordingly, the compositions of the invention find use in a variety of functional screens. The functional screens are used to elucidate the physiological role of the ubiquitin agents examined in the screen. Examples of functional screens are varied, and can include any of a variety of screens including cellular assays. In addition, the functional screens can include biochemical assays such as detecting in increase or decrease in a putative ubiquitin substrate or target molecule.

In any event, the functional screens include expressing in a cell system ubiquitin agents and determining an increase or decrease in a potential ubiquitin substrate or target molecule. That is, without being bound by theory, ubiquitination of target molecules targets the molecules for proteolysis. Thus, a decrease in the protein level of a potential ubiquitin substrate indicates that the ubiquitin agents are involved in ubiquitination of that substrate.

Conversely, the assay can be run in the opposite direction with a negative effector molecule. In this embodiment, a negative effector of a particular ubiquitin agent is introduced in a cell and an increase of a potential ubiquitin target molecule is examined. Again, because ubiquitin targets molecules for proteolysis, when ubiquitin agents are inhibited, for example with a dominant negative, the target molecules are not ubiquitinated and therefore are not targeted for degradation.

In a preferred embodiment, the present invention provides a method for performing functional deubiquitination screens. In a preferred embodiment, the method comprises contacting a cell with a negative effector of a ubiquitin agent and screening for an altered phenotype in the cell. By “negative effector” is meant a molecule known or believed to decrease the functional activity of a ubiquitin agent in a cell. The decrease in functional activity may arise via any mechanism, including through reduction of expression of the ubiquitin agent, either at the transcriptional or translational level (e.g., using siRNA or antisense RNA directed against nucleic acid encoding the ubiquitin agent), competition with an endogenous ubiquitin agent (e.g., using a dominant negative mutant of t he ubiquitin agent) or binding and, preferably, interfering with function of a ubiquitin agent (e.g., using a peptide, cyclic or linear, or other binding molecule such as a small organic molecule).

In an alternate embodiment, the methods include providing a cell culture, whose cells contain a library of nucleic acids comprising nucleic acids encoding at least one negative effector of ubiquitin agents. The invention further provides screening the cell culture for altered phenotype as compared to control cells, isolating those with altered phenotypes and identifying the negative effector of the ubiquitin agent(s) that resulted in the altered phenotype.

In one embodiment, the invention provides culturing cells expressing or over-expressing different ubiquitin agents and assaying a functional readout for the activity of the ubiquitin agents. Modulation of the functional readout indicates involvement of the ubiquitin agent in that pathway.

In a preferred general embodiment, the methods involve expressing a negative effector of a ubiquitin agent in a cell system and determining the effect of the variant ubiquitin agent in a functional assay. The functional assay may involve a cellular readout as described below, or may involve determining the amount of ubiquitin on a target protein. That is, the method involves measuring the amount of ubiquitin moiety attached to at least one of the following substrate molecules: a ubiquitin agent; a target protein; or a mono- or poly-ubiquitin moiety which is preferably attached to a ubiquitin agent or target protein.

Accordingly, the compositions of the invention find use in a variety of functional screens. The functional screens are used to elucidate the physiological role of the ubiquitin agent examined in the screen, i.e., to determine whether a particular ubiquitin agent is a modulator of a particular function. By “modulator” is meant the ability to enhance or inhibit, or increase or decrease a particular functional event. Such information provides instruction for the development of therapies for disease states associated with the function screened. In many instances, the negative effectors of the ubiquitin agents may serve as therapeutics themselves, or as models for the production of therapeutic molecules.

Examples of functional screens are varied, and can include any of a variety of screens including cellular assays. In addition, the functional screens can include biochemical assays such as detecting an increase or decrease in a putative ubiquitin substrate or target molecule.

In any event, in one embodiment the functional screens include expressing in a cell or cell population one or more ubiquitin agents or negative effectors thereof, and determining an increase or decrease in a potential ubiquitin substrate or target molecule.

The level of proteins can be examined in any of a variety of methods as are known to those of ordinary skill of the art. These methods include immunoblotting, or detecting labeled proteins, for example His-tagged proteins or radio-labeled proteins, and the like. In addition, protein identification can be accomplished by mass spectrometry. This is particularly useful when the identity of the proteins is unknown.

In a preferred embodiment, the functional screens include detecting a change in cell viability. That is, cells can be cultured expressing a negative effector of a ubiquitin agent, such as a dominant negative, or wild type ubiquitin agent. The cultures can be compared to control cultures and the level of cell viability examined. Cell viability can be determined by any of a variety of methods that are known to those of ordinary skill in the art.

In addition, cell cycle progression can be monitored as a function of expression of various wild type uniquitin agents or a negative effector of a ubiquitin agent. The cell cycle progression can be examined by methods known in the art as described in U.S. patent application Ser. No. 09/157,748, filed Sep. 21, 1998, which is expressly incorporated herein by reference.

Additional functional assays include screening for modulators of IgE as described in more detail in U.S. Ser. No. 09/076,624, filed May 12, 1998, U.S. Ser. No. 09/963,247, filed Sep. 25, 2001, U.S. Ser. No. 60/165,189, filed Nov. 12, 1999, U.S. Ser. No. 09/963,206, filed Sep. 25, 2001, and U.S. Ser. No. 09/966,976, filed Sep. 27, 2001, which are expressly incorporated herein by reference. Additional functional assays include screening for exocytosis modulators as set forth in U.S. Ser. No. 09/062,330, filed Apr. 17,1998, which is expressly incorporated herein by reference.

Additional functional assays include screening for modulators of T-cells and B-cells as set forth and U.S. Ser. No. 09/429,578, filed Oct. 28, 1999, which is expressly incorporated herein by reference.

Additional functional assays include screening for modulators of angiogenesis, macrophage activation, astrocyte differentiation. Preferred functional assays include but are not limited to cell cycle assays, cell proliferation assays, assays for apoptosis, assays for T-cell and B-cell activation, assays for macrophage and monocyte activation, assays for cell adhesion, assays for ostecloast differentiation, assays for cholesterol metabolism and assays for neurodegenerative disease. These assays are described as cited above and in more detail in the examples. All references are expressly incorporated herein by reference.

The functional assays of the present invention may be useful to screen a large number of cell types under a wide variety of conditions. In one embodiment, host cells are cells that are involved in disease states.

In a preferred embodiment, the present methods are useful in cancer applications. The ability to rapidly and specifically kill tumor cells is a cornerstone of cancer chemotherapy. In general, using the methods of the present invention, a ubiquitin agent or a negative effector of a ubiquitin agent can be introduced into any tumor cell (primary or cultured), and ubiquitin agents can thereby be identified which modulate apoptosis, cell death, loss of cell division or decreased cell growth. In an alternative embodiment, libraries encoding ubiquitin agents or putative negative effectors of a ubiquitin agent(s) can be introduced into any tumor cell (primary or cultured), and ubiquitin agents or negative effector(s) of ubiquitin agents can be identified which induce apoptosis, cell death, loss of cell division or decreased cell growth.

Alternatively, the methods of the present invention can be combined with other cancer therapeutics (e.g. drugs, such as taxol, or radiation) to sensitize the cells and thus induce rapid and specific apoptosis, cell death, loss of cell division or decreased cell growth after exposure to a secondary agent. Similarly, the present methods may be used in conjunction with known cancer therapeutics to screen for agonists to make the therapeutic more effective or less toxic. This is particularly preferred when the chemotherapeutic is very expensive to produce such as taxol. Other cancer applications are described in more detail in U.S. Ser. No. 09/800,770, filed Mar. 6, 2001, which is expressly incorporated herein by reference.

In a preferred embodiment, the present methods are useful in cardiovascular applications. In a preferred embodiment, cardiomyocytes may be screened for the prevention of cell damage or death in the presence of normally injurious conditions, including, but not limited to, the presence of toxic drugs (particularly chemotherapeutic drugs), for example, to prevent heart failure following treatment with adriamycin; anoxia, for example in the setting of coronary artery occlusion; and autoimmune cellular damage by attack from activated lymphoid cells (for example as seen in post viral myocarditis and lupus). Ubiquitin agents or negative effectors of ubiquitin agents can inserted into cardiomyocytes, which cells are subjected to the insult, and ubiquitin agents are identified which modulate any or all of: apoptosis; membrane depolarization (i.e. decrease arrythmogenic potential of insult); cell swelling; or leakage of specific intracellular ions, second messengers and activating molecules (for example, arachidonic acid and/or lysophosphatidic acid).

In a preferred embodiment, the present methods are used to screen for diminished arrhythmia potential in cardiomyocytes. The screens comprise the introduction of one or more ubiquitin agents or one or more negative effectors of ubiquitin agents into the cardiomycytes, followed by the application of arrythmogenic insults, thereby identifying ubiquitin agents that modulate specific depolarization of cell membrane. This may be detected using patch clamps, or via fluorescence techniques). Similarly, channel activity (for example, potassium and chloride channels) in cardiomyocytes could be regulated using the present methods in order to enhance contractility and prevent or diminish arrhythmias.

In a preferred embodiment, the present methods are used to screen for enhanced contractile properties of cardiomyocytes and diminish heart failure potential. The introduction of one or more ubiquitin agents, one or more negative effectors of ubiquitin agents, or libraries thereof, followed by measuring the rate of change of myosin polymerization/depolymerization using fluorescent techniques can be done. Ubiquitin agents may be identified that modulate this cellular electrochemical flux. An increase in the rate of change of this phenomenon can result in a greater contractile response of the entire myocardium, similar to the effect seen with digitalis.

In a preferred embodiment, the present methods are useful to identify agents that will regulate the intracellular and sarcolemmal calcium cycling in cardiomyocytes in order to prevent arrhythmias. Ubiquitin agents or negative effectors of ubiquitin agents are selected that regulate sodium-calcium exchange, sodium proton pump function, and regulation of calcium-ATPase activity.

In a preferred embodiment, the present methods are useful to identify ubiquitin agents that modulate embolic phenomena in arteries and arterioles leading to strokes (and other occlusive events leading to kidney failure and limb ischemia) and angina precipitating a myocardial infarct. For example, ubiquitin agents or negative effectors of ubiquitin agents are identified that will diminish the adhesion of platelets and leukocytes, and thus diminish the occlusion events. Adhesion in this setting can be inhibited by the ubiquitin agents, negative effectors, or libraries thereof of the invention being introduced into endothelial cells (quiescent cells, or activated by cytokines, i.e. IL-1, and growth factors, i.e. PDGF/EGF) by screening for ubiquitin agents or negative effectors of ubiquitin agents that induce either: 1) down regulation of adhesion molecule expression on the surface of the endothelial cells (binding assay); 2) blockade of adhesion molecule activation on the surface of these cells (signaling assay); or 3) release in an autocrine manner peptides that block receptor binding to the cognate receptor on the adhering cell.

Embolic phenomena can also be addressed by activating proteolytic enzymes on the cell surfaces of endothelial cells, and thus releasing active enzyme which can digest blood clots. Thus, delivery of the ubiquitin agents, negative effectors of ubiquitin agents, or libraries thereof, of the invention to endothelial cells is done, followed by standard fluorogenic assays, which will allow monitoring of proteolytic activity on the cell surface towards a known substrate. Ubiquitin agents can then be identified which modulate activation of specific enzymes towards specific substrates.

In a preferred embodiment, arterial inflammation in the setting of vasculitis and post-infarction can be regulated by decreasing the chemotactic responses of leukocytes and mononuclear leukocytes. This can be accomplished by blocking chemotactic receptors and their responding pathways on these cells. Ubiquitin agents, negative effectors of ubiquitin agents, or libraries thereof, can be inserted into these cells, and the chemotactic response to diverse chemokines (for example, to the IL-8 family of chemokines, RANTES) determined in cell migration assays.

In a preferred embodiment, arterial restenosis following coronary angioplasty can be controlled by regulating the proliferation of vascular intimal cells and capillary and/or arterial endothelial cells. Ubiquitin agents, negative effectors of ubiquitin agents, or libraries thereof, can be inserted into these cell types and their proliferation in response to specific stimuli monitored.

The control of capillary and blood vessel growth is an important goal in order to promote increased blood flow to ischemic areas (growth), or to cut-off the blood supply (angiogenesis inhibition) of tumors. Ubiquitin agents, negative effectors of ubiquitin agents, or libraries thereof, can be inserted into capillary endothelial cells and their growth monitored. Stimuli such as low oxygen tension and varying degrees of angiogenic factors can regulate the responses, and peptides isolated that produce the appropriate phenotype. Screening for modulation of vascular endothelial cell growth factor, important in angiogenesis, would also be useful.

In a preferred embodiment, the present methods are useful in screening for modulators of atherosclerosis producing mechanisms to find ubiquitin agents that regulate LDL and HDL metabolism. Ubiquitin agents, negative effectors of ubiquitin agents, or libraries thereof, can be inserted into the appropriate cells (including hepatocytes, mononuclear leukocytes, endothelial cells) and ubiquitin agents can be identified which modulate release of LDL or synthesis of LDL, or conversely release of HDL or synthesis of HDL. Ubiquitin agents, negative effectors of ubiquitin agents, or libraries thereof, can also be used to identify ubiquitin wagents that modulate the production of oxidized LDL, which has been implicated in atherosclerosis and isolated from atherosclerotic lesions. Modulation could occur by altering its expression, modulating reducing systems or enzymes, or affecting the activity or production of enzymes implicated in production of oxidized LDL, such as 1 5-lipoxygenase in macrophages.

In a preferred embodiment, the present methods are used in screens to identify ubiquitin agents that regulate obesity via the control of food intake mechanisms or the responses of receptor signaling pathways that regulate metabolism. Identification of ubiquitin agents or negative effectors of ubiquitin agents that regulate or inhibit the responses of neuropeptide Y (NPY), cholecystokinin and galanin receptors, are particularly desirable. Candidate libraries can be inserted into cells that have these receptors cloned into them, and modulatory molecules selected.

In a preferred embodiment, the present methods are useful in neurobiology applications. Ubiquitin agents, negative effectors of ubiquitin agents, or libraries thereof, may be used for screening for modulators of neuronal apoptotis, with an eye to preserving neuronal function and preventing of neuronal death. Initial screens would be done in cell culture. One application would include determining modulation of neuronal death, by apoptosis, in cerebral ischemia resulting from stroke. Apoptosis is known to be blocked by neuronal apoptosis inhibitory protein (NAIP); screens for its upregulation, down regulation, or affecting any coupled step could identify molecules which selectively modulate neuronal apoptosis. Other applications include neurodegenerative diseases such as Alzheimer's disease and Huntington's disease.

In a preferred embodiment, the present methods are useful in bone biology applications. Osteoclasts are known to play a key role in bone remodeling by breaking down “old” bone, so that osteoblasts can lay down “new” bone. In osteoporosis one has an imbalance of this process. Osteoclast overactivity can be regulated by inserting ubiquitin agents, negative effectors of ubiquitin agents, or libraries thereof, into these cells, and then looking for molecules that result in: 1) altrered processing of collagen by these cells; 2) altered pit formation on bone chips; and 3) altered release of calcium from bone fragments.

The present methods may also be used to screen for agonists of bone morphogenic proteins, hormone mimetics to stimulate, regulate, or enhance new bone formation (in a manner similar to parathyroid hormone and calcitonin, for example). These have use in osteoporosis, for poorly healing fractures, and to accelerate the rate of healing of new fractures. Furthermore, cell lines of connective tissue origin can be treated with ubiquitin agents, negative effectors of ubiquitin agents, or libraries thereof, and screened for their growth, proliferation, collagen stimulating activity, and/or proline incorporating ability on the target osteoblasts. Alternatively, ubiquitin agents, negative effectors of ubiquitin agents, or libraries thereof, can be expressed directly in osteoblasts or chondrocytes and screened for modulation of production of collagen or bone.

In a preferred embodiment, the present methods are useful in skin biology applications. Keratinocyte responses to a variety of stimuli may result in psoriasis, a proliferative change in these cells. Ubiquitin agents, negative effectors of ubiquitin agents, or libraries thereof, can be inserted into cells removed from active psoriatic plaques, and candidate ubiquitin agents or dominant negative ubiquitin agents isolated which modulate the rate of growth of these cells.

In a preferred embodiment, the present methods are useful in the identification of modulators of regulation of keloid formation (i.e. excessive scarring). Ubiquitin agents, negative effectors of ubiquitin agents, or libraries thereof, inserted into skin connective tissue cells isolated from individuals with this condition, can identify ubiquitin agents that modulate proliferation, collagen formation, or proline incorporation. Results from this work can be extended to treat the excessive scarring that also occurs in burn patients. If a common modulator is found in the context of the keloid work, then it can be used widely in a topical manner to diminish scarring post burn.

Similarly, wound healing for diabetic ulcers and other chronic “failure to heal” conditions in the skin and extremities can be regulated by providing additional growth signals to cells which populate the skin and dermal layers. Growth factor mimetics may in fact be very useful for this condition. Ubiquitin agents, negative effectors of ubiquitin agents, or libraries thereof, can be inserted into skin connective tissue cells, and ubiquitin agents identified which modulate the growth of these cells under “harsh” conditions, such as low oxygen tension, low pH, and the presence of inflammatory mediators.

Cosmeceutical applications of the present invention include the control of melanin production in skin melanocytes. A naturally occurring peptide, arbutin, is a tyrosine hydroxylase inhibitor, a key enzyme in the synthesis of melanin. Ubiquitin agents, negative effectors of ubiquitin agents, or libraries thereof, can be inserted into melanocytes and known stimuli that increase the synthesis of melanin applied to the cells. Candidate ubiquitin agents can be identified that modulate the synthesis of melanin under these conditions.

In a preferred embodiment, the present methods are useful in endocrinology applications. The delivery methods described herein can be applied broadly to any endocrine, growth factor, cytokine or chemokine network which involves a signaling peptide or protein that acts in either an endocrine, paracrine or autocrine manner that binds or dimerizes a receptor and activates a signaling cascade that results in a known phenotypic or functional outcome. The methods are applied so as to identify a ubiquitin agent that modulates the desired hormone (i.e., insulin, leptin, calcitonin, PDGF, EGF, EPO, GMCSF, IL1-17, mimetics) or its action by either modulating the release of the hormone, modulating its binding to a specific receptor or carrier protein (for example, CRF binding protein), or modualting the intracellular responses of the specific target cells to that hormone. Identification of ubiquitin agents which modulate the expression or release of hormones from the cells which normally produce them could have broad applications to conditions of hormonal deficiency.

In a preferred embodiment, the present methods are useful in infectious disease applications. Viral latency (herpes viruses such as CMV, EBV, HBV, and other viruses such as HIV) and their reactivation are a significant problem, particularly in immunosuppressed patients (patients with AIDS and transplant patients). The ability to block the reactivation and spread of these viruses is an important goal. Cell lines known to harbor or be susceptible to latent viral infection can be infected with the specific virus, and then stimuli applied to these cells which have been shown to lead to reactivation and viral replication. This can be followed by measuring viral titers in the medium and scoring cells for phenotypic changes. Ubiquitin agents, negative effectors of ubiquitin agents, or libraries thereof, can then be introduced into these cells under the above conditions, and agents identified which modulate the growth and/or release of the virus. As with chemotherapeutics, these experiments can also be done with drugs which are only partially effective towards this outcome, and bioactive peptides isolated which enhance the virucidal effect of these drugs. Agents may also be tested for the ability to block some aspect of viral assembly, viral replication, entry or infectious cycle. Additional disclosure directed to reduction of viral infection, including HIV, is set forth in U.S. Ser. No. 09/800,770, filed Mar. 6, 2001, which is expressly incorporated herein by reference.

In a preferred embodiment, the present invention finds use with infectious organisms. Intracellular organisms such as mycobacteria, listeria, salmonella, pneumocystis, yersinia, leishmania, T. cruzi, can persist and replicate within cells, and become active in immunosuppressed patients. There are currently drugs on the market and in development which are either only partially effective or ineffective against these organisms. Ubiquitin agents, negative effectors of ubiquitin agents, or libraries thereof, can be inserted into specific cells infected with these organisms (pre- or post-infection), and ubiquitin agents identified which modulate the intracellular destruction of these organisms in a manner analogous to intracellular “antibiotic peptides” similar to magainins. In addition ubiquitin agents can be identified which modulate the cidal properties of drugs already under investigation which have insufficient potency by themselves, but when combined with a specific peptide from a candidate library, are dramatically more potent through a synergistic mechanism. Finally, ubiquitin agents can be identified which affect the metabolism of these intracellular organisms, with an eye towards terminating their intracellular life cycle by inhibiting a key organismal event.

Antibiotic drugs that are widely used have certain dose dependent, tissue specific toxicities. For example renal toxicity is seen with the use of gentamicin, tobramycin, and amphotericin; hepatotoxicity is seen with the use of INH and rifampin; bone marrow toxicity is seen with chloramphenicol; and platelet toxicity is seen with ticarcillin, etc. These toxicities limit their use. ubiquitin agents, negative effectors of ubiquitin agents, or libraries thereof, can be introduced into the specific cell types where specific changes leading to cellular damage or apoptosis by the antibiotics are produced, and ubiquitin agents can be identified that modulate sensitivity, when these cells are treated with these specific antibiotics.

Furthermore, the present invention finds use in screening for ubiquitin agents that modulate antibiotic transport mechanisms. The rapid secretion from the blood stream of certain antibiotics limits their usefulness. For example penicillins are rapidly secreted by certain transport mechanisms in the kidney and choroid plexus in the brain. Probenecid is known to block this transport and increase serum and tissue levels. Ubiquitin agents, negative effectors of ubiquitin agents, or libraries thereof, can be inserted into specific cells derived from kidney cells and cells of the choroid plexus known to have active transport mechanisms for antibiotics. Ubiquitin agents can then be identified which block the active transport of specific antibiotics and thus extend the serum halflife of these drugs.

In a preferred embodiment, the present methods are useful in drug toxicities and drug resistance applications. Drug toxicity is a significant clinical problem. This may manifest itself as specific tissue or cell damage with the result that the drug's effectiveness is limited. Examples include myeloablation in high dose cancer chemotherapy, damage to epithelial cells lining the airway and gut, and hair loss. Specific examples include adriamycin induced cardiomyocyte death, cisplatinin-induced kidney toxicity, vincristine-induced gut motility disorders, and cyclosporin-induced kidney damage. Ubiquitin agents, negative effectors of ubiquitin agents, or libraries thereof, can be introduced into specific cell types with characteristic drug-induced phenotypic or functional responses, in the presence of the drugs, and ubiquitin agents identified which modulate toxicity in the specific cell-type when exposed to the drug. These effects may manifest as modulating the drug induced apoptosis of the cell of interest, thus initial screens will determine relative survival of the cells in the presence of high levels of drugs or combinations of drugs used in combination chemotherapy.

Drug toxicity may be due to a specific metabolite produced in the liver or kidney which is highly toxic to specific cells, or due to drug interactions in the liver which block or enhance the metabolism of an administered drug. Ubiquitin agents, negative effectors of ubiquitin agents, or libraries thereof, can be introduced into liver or kidney cells following the exposure of these cells to the drug known to produce the toxic metabolite. Ubiquitin agents can be identified which alter how the liver or kidney cells metabolize the drug, and specific ubiquitin agents identified which modulate the generation of a specific toxic metabolite. The generation of the metabolite can be followed by mass spectrometry, and phenotypic changes can be assessed by microscopy. Such a screen can also be done in cultured hepatocytes, cocultured with readout cells which are specifically sensitive to the toxic metabolite. Applications include reversible (to limit toxicity) inhibitors of enzymes involved in drug metabolism.

Multiple drug resistance, and hence tumor cell selection, outgrowth, and relapse, leads to morbidity and mortality in cancer patients. Ubiquitin agents, negative effectors of ubiquitin agents, or libraries thereof, can be introduced into tumor cell lines (primary and cultured) that have demonstrated specific or multiple drug resistance. Ubiquitin agents can then be identified which modulate drug sensitivity when the cells are exposed to the drug of interest, or to drugs used in combination chemotherapy. The readout can be the onset of apoptosis in these cells, membrane permeability changes, the release of intracellular ions and fluorescent markers. The cells in which multidrug resistance involves membrane transporters can be preloaded with fluorescent transporter substrates, and selection carried out for ubiquitin agents which modulate the normal efflux of fluorescent drug from these cells.

Ubiquitin agents, negative effectors of ubiquitin agents, and in particular libraries thereof, are suited to screening for ubiquitin agents which modulate poorly characterized or recently discovered intracellular mechanisms of resistance or mechanisms for which few or no chemosensitizers currently exist, such as mechanisms involving LRP (lung resistance protein). This protein has been implicated in multidrug resistance in ovarian carcinoma, metastatic malignant melanoma, and acute myeloid leukemia. Particularly interesting examples include screening for ubiquitin agents which modulate more than one important resistance mechanism in a single cell, which occurs in a subset of the most drug resistant cells, which are also important targets. Applications would include screening for ubiquitin agent modulators of both MRP (multidrug resistance related protein) and LRP for treatment of resistant cells in metastatic melanoma, for modulators of both p-glycoprotein and LRP in acute myeloid leukemia, and for modulation (by any mechanism) of all three proteins for treating pan-resistant cells.

In a preferred embodiment, the present methods are useful in improving the performance of existing or developmental drugs. First pass metabolism of orally administered drugs limits their oral bioavailability, and can result in diminished efficacy as well as the need to administer more drug for a desired effect. Reversible inhibitors of enzymes involved in first pass metabolism may thus be a useful adjunct enhancing the efficacy of these drugs. First pass metabolism occurs in the liver, thus inhibitors of the corresponding catabolic enzymes may enhance the effect of the cognate drugs. Reversible inhibitors would be delivered at the same time as, or slightly before, the drug of interest. Screening of ubiquitin agents, negative effectors of ubiquitin agents, or libraries thereof, in hepatocytes for modulators (by any mechanism, such as protein downregulation as well as a direct inhibition of activity) of particularly problematical isozymes would be of interest. These include the CYP3A4 isozymes of cytochrome P450, which are involved in the first pass metabolism of the anti-HIV drugs saquinavir and indinavir. Other applications could include reversible inhibitors of UDP-glucuronyltransferases, sulfotransferases, N-acetyltransferases, epoxide hydrolases, and glutathione S-transferases, depending on the drug. Screens would be done in cultured hepatocytes or liver microsomes, and could involve antibodies recognizing the specific modification performed in the liver, or co-cultured readout cells, if the metabolite had a different bioactivity than the untransformed drug. The enzymes modifying the drug would not necessarily have to be known, if screening was for lack of alteration of the drug.

In a preferred embodiment, the present methods are useful in immunobiology, inflammation, and allergic response applications. Selective regulation of T lymphocyte responses is a desired goal in order to modulate immune-mediated diseases in a specific manner. Ubiquitin agents, negative effectors of ubiquitin agents, or libraries thereof, can be introduced into specific T cell subsets (TH1, TH2, CD4+, CD8+, and others) and the responses which characterize those subsets (cytokine generation, cytotoxicity, proliferation in response to antigen being presented by a mononuclear leukocyte, and others) modified by members of the library. Ubiquitin agents can be identified which modulate the known T cell subset physiologic response. This approach will be useful in any number of conditions, including: 1) autoimmune diseases where one wants to induce a tolerant state (select a peptide that inhibits T cell subset from recognizing a self-antigen bearing cell); 2) allergic diseases where one wants to decrease the stimulation of IgE producing cells (select peptide which blocks release from T cell subsets of specific B-cell stimulating cytokines which induce switch to IgE production); 3) in transplant patients where one wants to induce selective immunosuppression (select peptide that diminishes proliferative responses of host T cells to foreign antigens); 4) in lymphoproliferative states where one wants to inhibit the growth or sensitize a specific T cell tumor to chemotherapy and/or radiation; 5) in tumor surveillance where one wants to inhibit the killing of cytotoxic T cells by Fas ligand bearing tumor cells; and 5) in T cell mediated inflammatory diseases such as Rheumatoid arthritis, Connective tissue diseases (SLE), Multiple sclerosis, and inflammatory bowel disease, where one wants to inhibit the proliferation of disease-causing T cells (promote their selective apoptosis) and the resulting selective destruction of target tissues (cartilage, connective tissue, oligodendrocytes, gut endothelial cells, respectively).

Regulation of B cell responses will permit a more selective modulation of the type and amount of immunoglobulin made and secreted by specific B cell subsets. Ubiquitin agents, negative effectors of ubiquitin agents, or libraries thereof, can be inserted into B cells and ubiquitin agents identified which modulate the release and synthesis of a specific immunoglobulin. This may be useful in autoimmune diseases characterized by the overproduction of auto antibodies and the production of allergy causing antibodies, such as IgE. Ubiquitin agents can also be identified which inhibit or enhance the binding of a specific immunoglobulin subclass to a specific antigen either foreign of self. Finally, ubiquitin agents can be identified which inhibit the binding of a specific immunoglobulin subclass to its receptor on specific cell types.

Similarly, ubiquitin agents which affect cytokine production may be identified, generally using two cell systems. For example, cytokine production from macrophages, monocytes, etc. may be evaluated. Similarly, deubiquitiniating agents which modulate cytokines, for example erythropoetin and IL1-17, may be identified.

Antigen processing by mononuclear leukocytes (ML) is an important early step in the immune system's ability to recognize and eliminate foreign proteins. Ubiquitin agents, negative effectors of ubiquitin agents, or libraries thereof, can be inserted into ML cell lines and agents selected which alter the intracellular processing of foreign peptides and sequence of the foreign peptide that is presented to T cells by MLs on their cell surface in the context of Class II MHC. One can look for dubiquitinating agents, negative effectors of ubiquitin agents, or libraries thereof, that affect responses of a particular T cell subset (for example, the peptide would in fact work as a vaccine). This approach could be used in transplantation, autoimmune diseases, and allergic diseases.

The release of inflammatory mediators (cytokines, leukotrienes, prostaglandins, platelet activating factor, histamine, neuropeptides, and other peptide and lipid mediators) is a key element in maintaining and amplifying aberrant immune responses. Ubiquitin agents, negative effectors of ubiquitin agents, or libraries thereof, can be inserted into MLs, mast cells, eosinophils, and other cells participating in a specific inflammatory response, and ubiquitin agents identifies that modulate the release and binding to the cognate receptor of each of these types of mediators.

In one embodiment wherein a library is screened, the method further comprises isolating at least one altered cell with said altered phenotype. Methods of isolating cells are known in the art and include, but are not limited to, FACS analysis and isolation, growth on selective medium, clonal isolation of cells and the like. In general, once the cell with the altered phenotype is identified, the cell(s) is then isolated for further analysis, e.g. to determine which ubiquitin agent variant resulted in the altered phenotype. Accordingly, the method further comprises identifying said variant agent in said altered cell. That is, once the cell(s) with the altered phenotype is identified and isolated, the nucleic acid encoding the ubiquitin agents or negative effector of a ubiquitin agent is identified. This is accomplished by isolating from the cellular DNA the insert encoding the ubiquitin agent variant. Preferably this is performed by PCR.

It is understood by the skilled artisan that the steps of the assays provided herein can vary in order. It is also understood, however, that while various options (of compounds, properties selected or order of steps) are provided herein, the options are also each provided individually, and can each be individually segregated from the other options provided herein. Moreover, steps which are obvious and known in the art that will increase the sensitivity of the assay are intended to be within the scope of this invention. For example, there may be additionally washing steps, blocking steps, etc.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are expressly incorporated by reference in their entirety.

### EXAMPLES

### Example 1

### A549, HUVEC, HBEC ICAM (CD54) Induction Assay

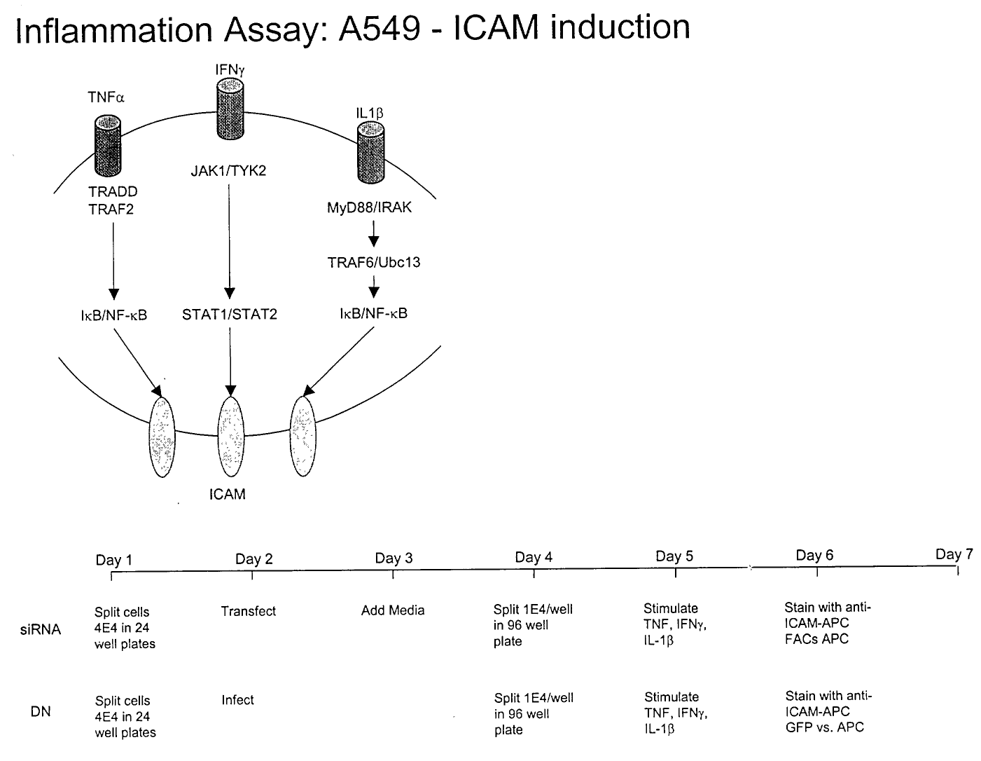

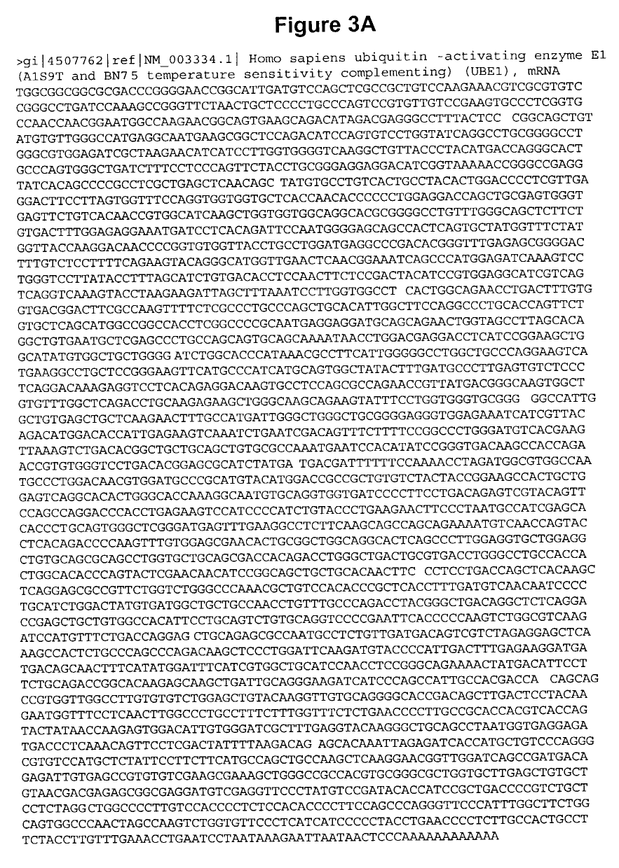

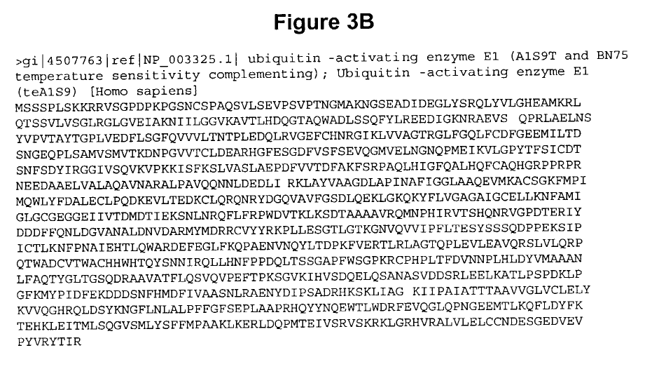

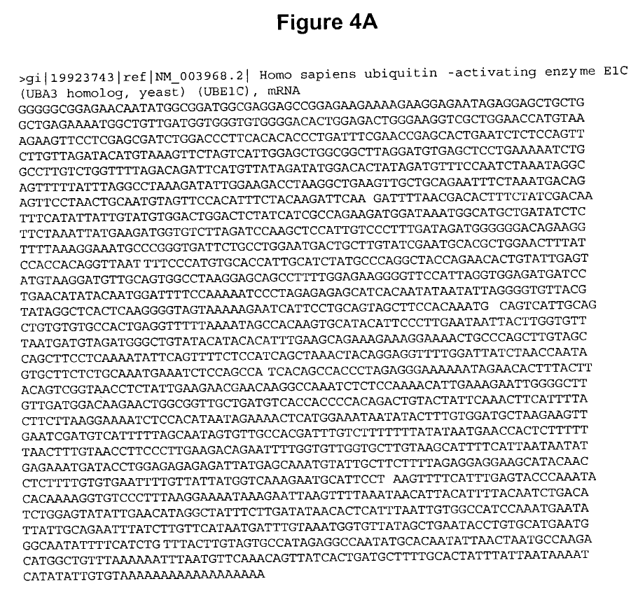

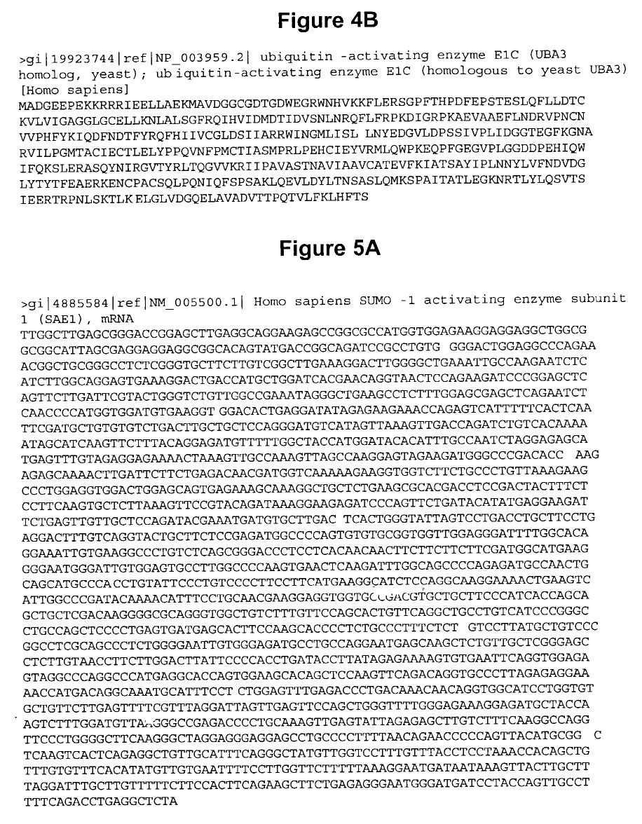

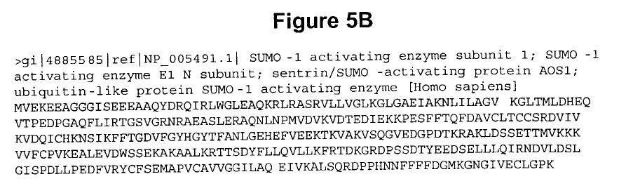

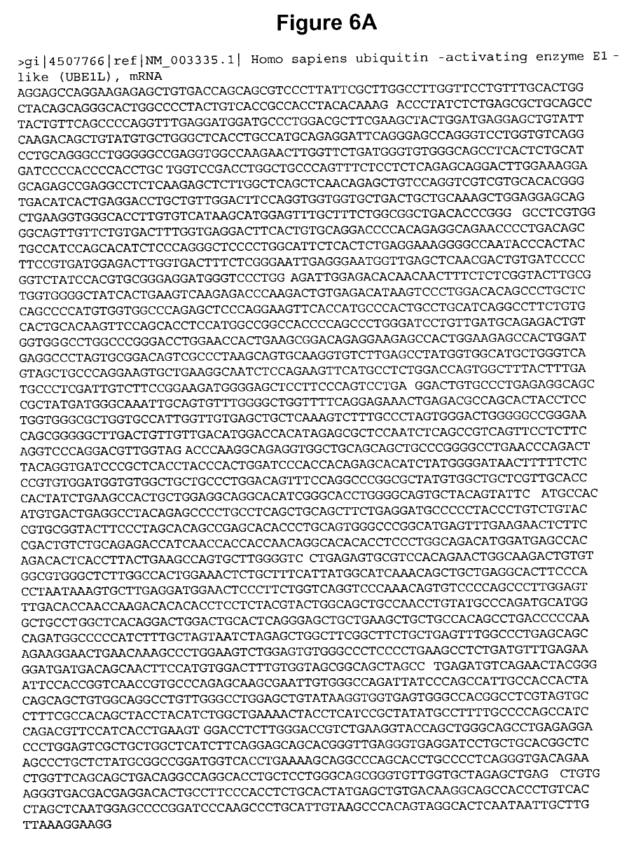

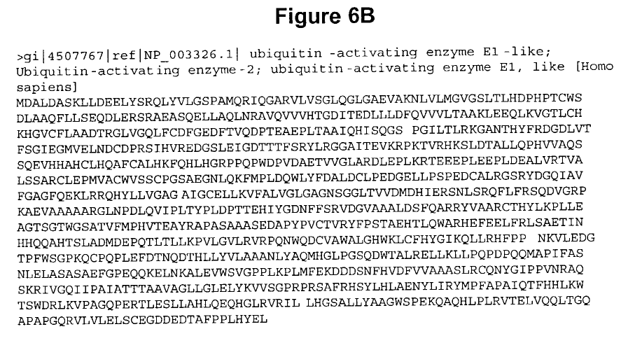

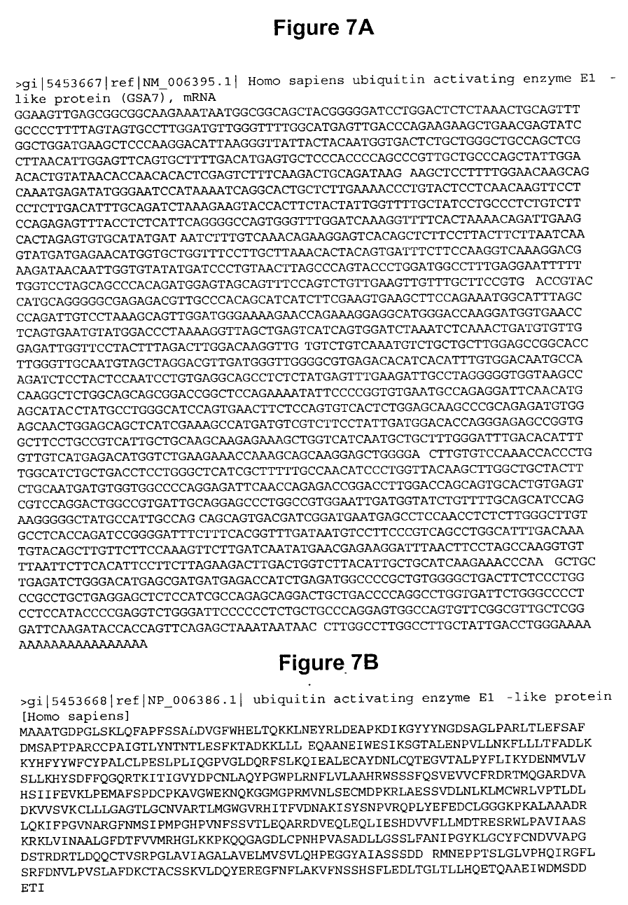

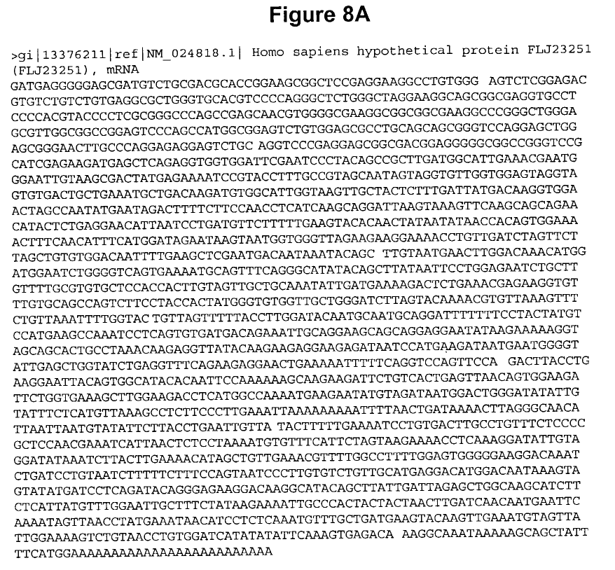

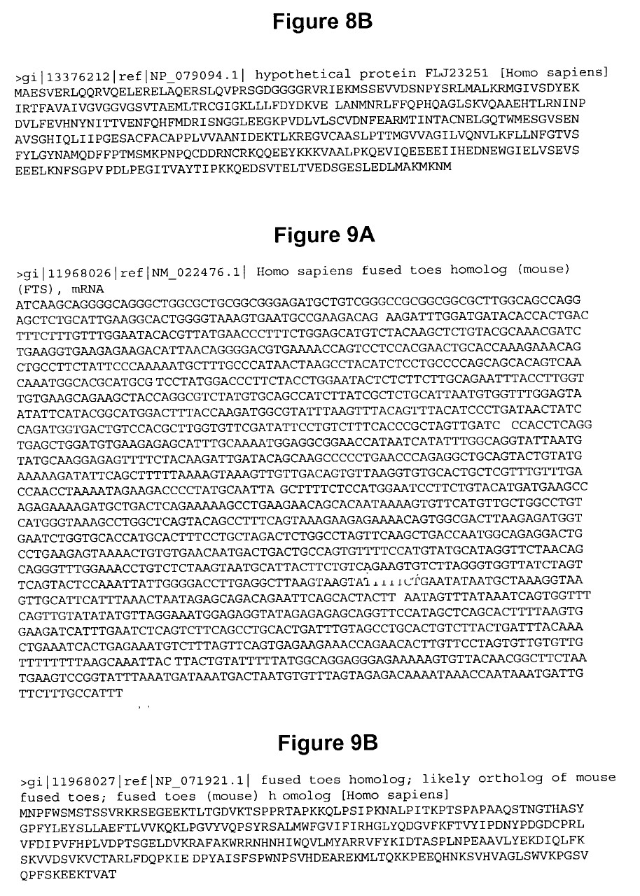

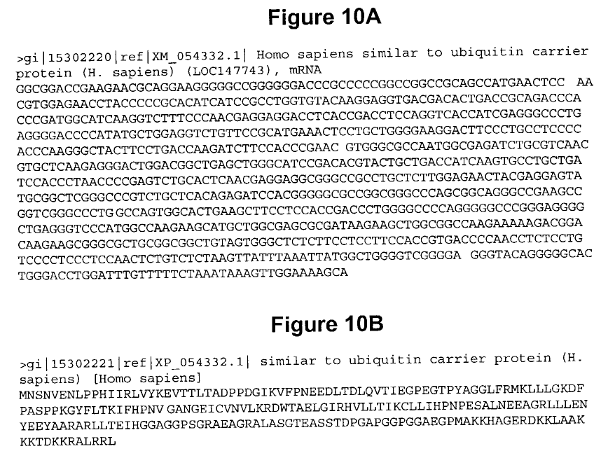

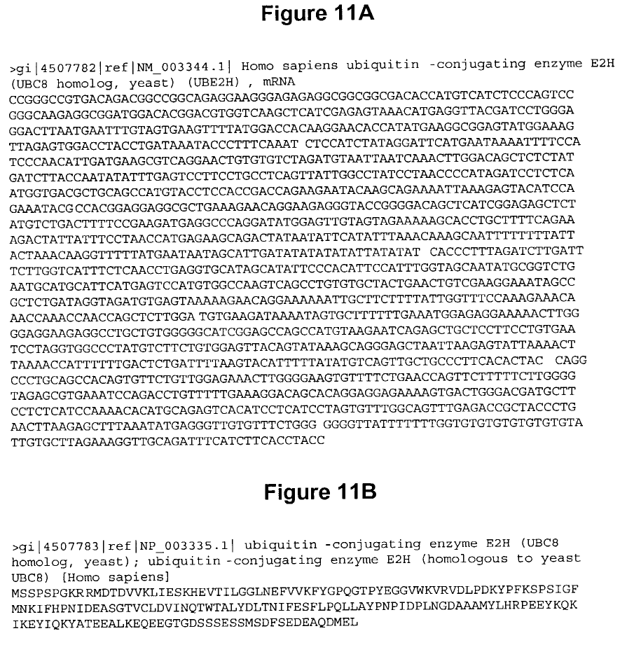

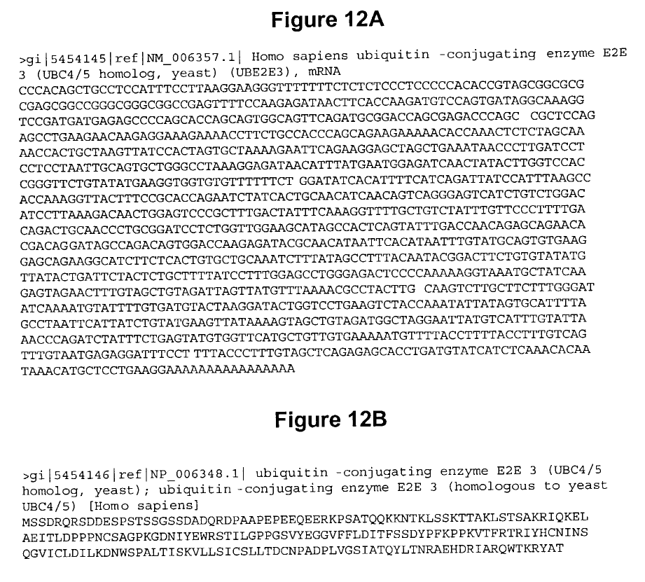

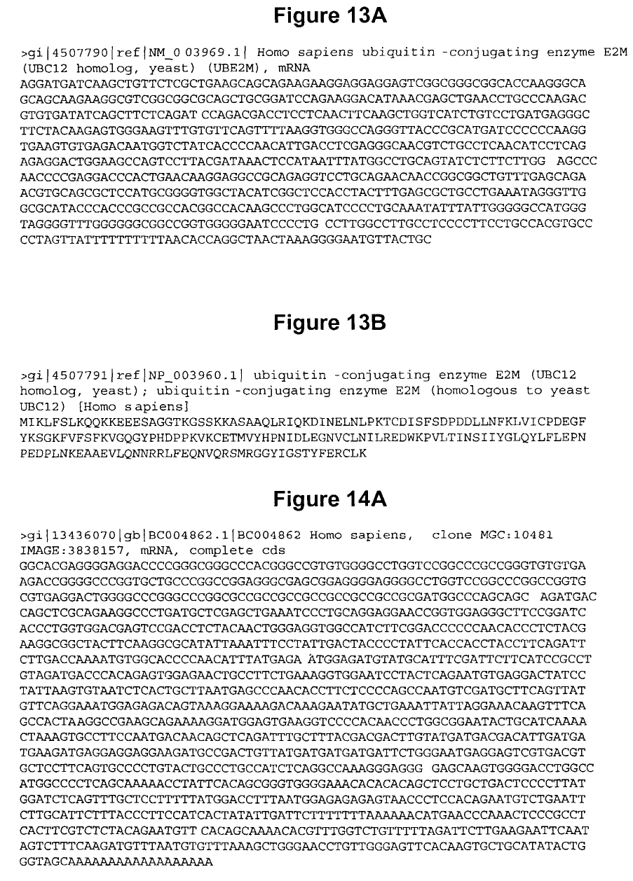

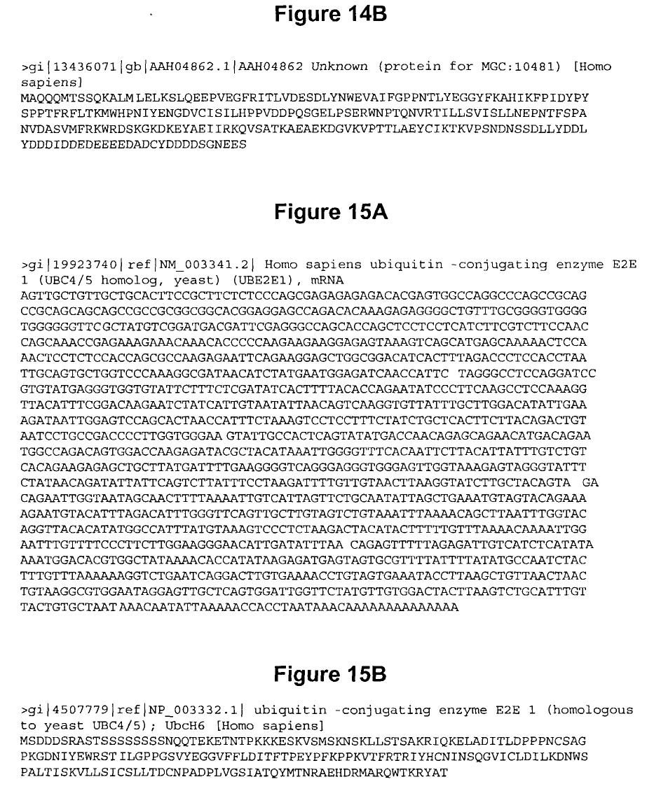

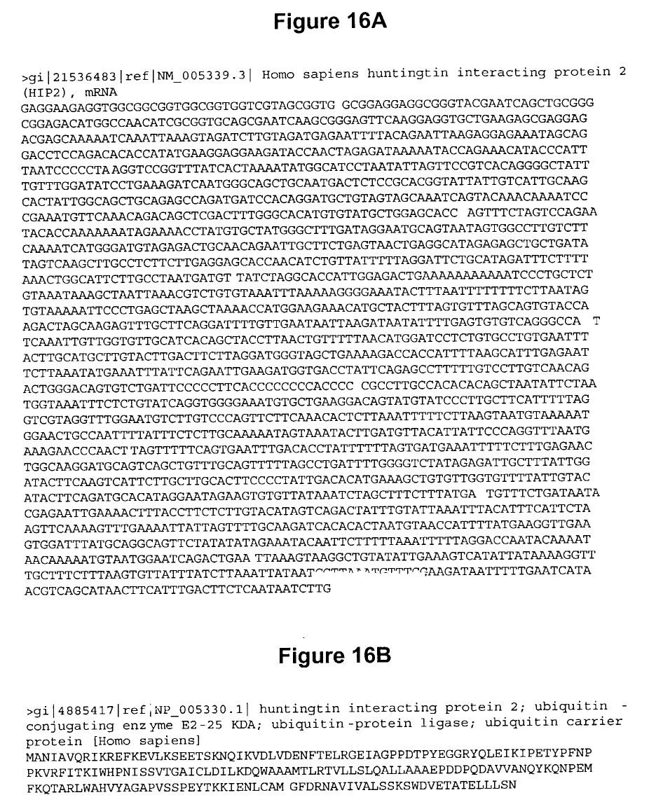

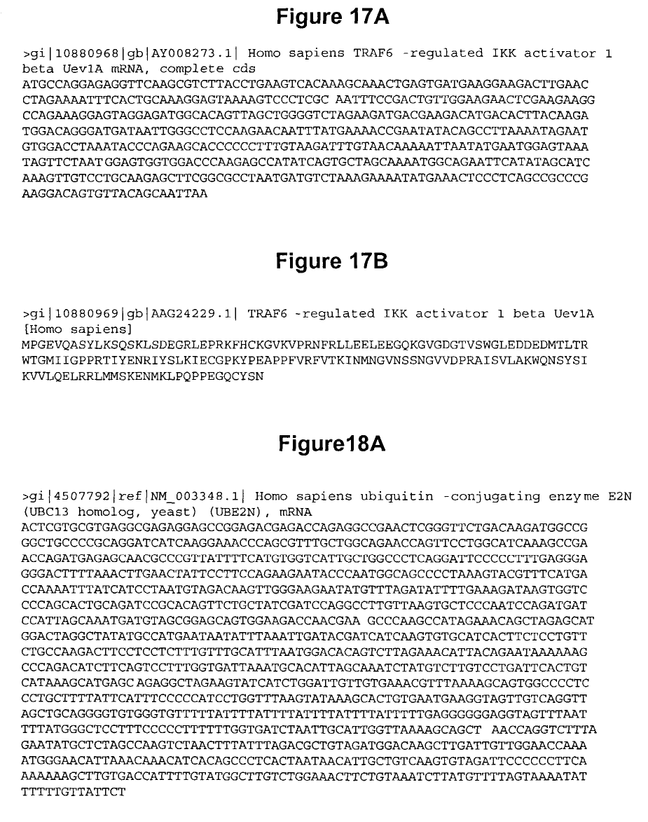

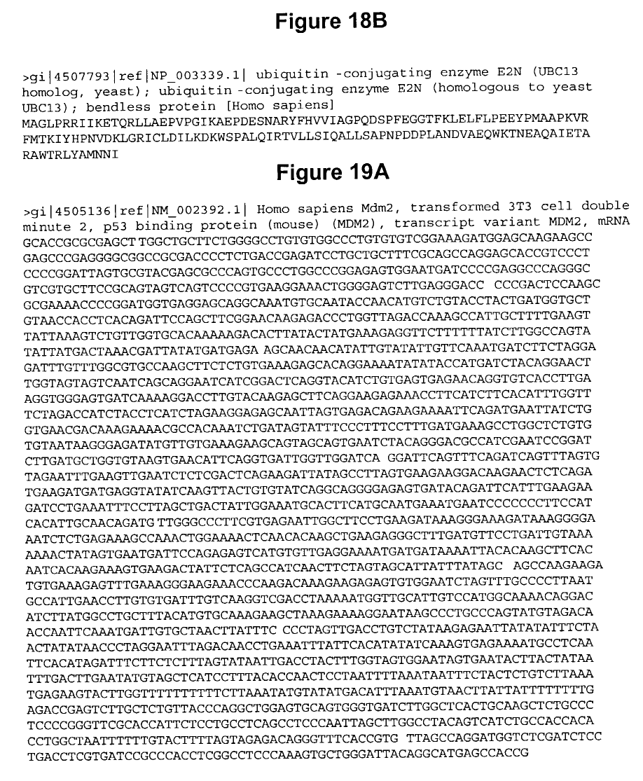

The ICAM upregulation assay models the inflammatory process and cytokine signaling. ICAM is an adhesion molecule that is expressed on the surface of cells at local sites of inflammation. ICAM expression is induced in the presence of various cytokines such as IL-1β, TNFα, and IFNγ. Each cytokine acts through different signaling molecules therefore this assay can delineate the specificity of a particular genetic effector (i.e. siRNA or a dominant interfering mutant) (see FIG. 2).

Day1:

Split cells (A549, HBEC, or HUVEC) cells 4.5×10⁴ in a 24 well plate in the appropriate media and incubate at 37° C., 5% C02.

Day 2: Cells should be 40-50% Confluent

siRNA

Transfect siRNA with oligofectamine. Pipette out the media and replace it with 500 uL of fresh media. Mix 3uL of 20 uM siRNA duplexes with 50 uL of Optimem media. Add 3 uL of oligofectamine to 12 uL Optimen. Wait 7-10 minutes. Combine the two solutions and gently pipette up and down 3 times. Wait 20-25 minutes. Add 32 uL of Optimen to adjust the volume to 100 uL. Add the entire mixture to the cells.

Retroviral

Infect cells using a standard spin infection protocol.

Day 3: Add 0.5 mL of Fresh Media

Day 4:

Wash cells in 1mL PBS, remove PBS and add 100 uL of Trypsin/EDTA. 5 min later add 100 uL of FK12. Pipette 4× up and down then transfer the cells to a V-bottom 96 well plate. Spin down at 1200 rpm for 3 min. Resuspend in 200 uL of fresh media. Count representative wells by hemocytometer then compute the average cells/mi. Plate 1.5×10⁴ cells/well in a 96 well plate, the total final volume is 50 uL.

Day 5:

Add 50 uL of a 2× cytokine mixture; the final concentrations of recombinant IL-1α, TNFβ, and IFNγ should be 75 ng/mL. All cytokines can be purchased from Peprotech as a lyophilized powder.

Day 6: Stain cells and FACs analysis

Rinse the cells 1×200 uL PBS. Add 50 uL of Trypsin/EDTA-lncubate 5 min at 37° C. Add 150 uL of PBS-2% FCS-Pipette up and down 5× and transfer to a V-bottom 96 well plate. Spin down and wash lx in 200 uL PBS-EDTA, remove solution. Add 25 uL of a 1:7 dilution of ICAM-APC (Pharmingen). Pipette up and down gently 4× to resuspend the cells. Incubate in the dark for 15 min at 4° C. Add 175 uL of PBS-2%FCS. Spin down at 2000 rpm for 30 sec. Wash once with 200 uL PBS-2%FCS. Add 150 uL of PBS-2%, resuspend the cells, then transfer to cluster tubes.

Perform FACS analysis on FL4-APC for siRNA analysis, FL4-APC vs. FL1-GFP for retroviral IRES or GFP-fusion analysis.

Results

As shown in the following tables, when various E1, E2 or E3 siRNA variants were introduced into the cells, ICAM induction in response to different cytokines was modulated. This demonstrates that the molecule targeted by the siRNA is involved in cytokine induction of ICAM.

TABLE 4
Summary of ICAM data with E1 variants

| Gene | ICAM | ICAM | ICAM |
|---|---|---|---|
| (with siRNA) | IFNg | IL-1b | TNF |
| E1.1 | NE | NE | NE |
| E1.4 | | | |
| E1.2 | NE | INH | NE |
| E1.3 | NE | INH | INH |
| E1.5 | | | |
| E1.6 | | | |
| E1.7 | | | |
| E1.8 | | | |
| E1.9 | | | |
| E1.10 | | | |
| E1.11 | | | |
| E1.12 | | | |
| E1.13 | | | |
| E1.14 | | | |

NE - not enhanced
INH - inhibited
ENH - enhanced

TABLE 5
Summary of ICAM data with E2 variants

| Gene | ICAM | ICAM | ICAM |
|---|---|---|---|
| (with siRNA) | TNF | FNg | IL-1b |
| E2.1 | NE | NE | NE |
| E2.15 | | | |
| E2.2 | NE | NE | NE |
| E2.16 | | | |
| E2.17 | | | |
| E2.3 | NE | NE | INH |
| E2.4 | NE | NE | NE |
| E2.18 | | | |
| E2.5 | NE | NE | NE |
| E2.19 | | | |
| UBE2D3 | | | |
| E2.20 | | | |
| E2.21 | | | |
| E2.6 | ENH | ENH | ENH |
| E2.22 | | | |
| E2.23 | | | |
| E2.7 | INH | INH | INH |
| E2.8 | NE | NE | NE |
| E2.9 | ENH | NE | NE |
| E2.10 | NE | NE | NE |
| E2.24 | | | |
| E2.11 | NE | INH | NE |
| E2.12 | NE | ENH | NE |
| E2.13 | NE | NE | NE |
| E2.25 | | | |
| E2.14 | NE | NE | INH |

TABLE 6
Summary of ICAN data with E3 variants

| Gene | ICAM | ICAM | ICAM |
|---|---|---|---|
| (with siRNA) | TNFa | IFNg | IL1b |
| E3.4 | NE | NE | ENH |
| E3.5 | NE | ENH | NE |
| E3.1 | ENH | ENH | ENH |
| E3.3 | NE | NE | INH |

### Example 2

### Jurkat and BJAB Activation Protocols

T/B Cell CD69 assay: For CD69 upregulation experiments, tTA-BJAB or tTA-Jurkat cells were split to 2.5×105 cells/ml 24 hours prior to stimulation. Cells were spun and resuspended at 5×10⁵ cells/ml in fresh complete RPMI medium in the presence of 0.3 ug/ml anti-lgM F(ab′)2 (Jackson lmmunoresearch), 300 ng/ml C305 (anti-Jurkat clonotypic TCR (19)) or 5ng/ml PMA for 16-20 hours at 37° C. Jurkat-N or tTA-BJAB cells were then stained with an APC-conjugated mouse monoclonal anti-human CD69 antibody (Caltag) at 4° C. for 30 minutes and analyzed using a Facscalibur instrument (Becton Dickinson) with Cellquest software.

T cell CD28RE-RFP assay: tTA-Jurkat cells stably transfected with a CD28RE/AP-driven RFP construct were split to 2.5×10⁵ cells/ml 24 hours prior to stimulation. Cells were spun and resuspended at 5×10⁵ cells/ml in fresh complete RPMI medium in the presence of platecoated 300 ng/ml C305 (anti-Jurkat clonotypic TCR (19)) plus 1 ug/ml a-CD28, or 5ng/ml PMA plus 1 uM lonmycin for 16-20 hours at 37° C. Jurkat-N cells were then analyzed using a Facscalibur instrument (Becton Dickinson) with Cellquest software (data not shown).

### Example 3

### LDL-Receptor Upregulation

This assay measures cytokine induced LDL-Receptor expression on HepG2 cells. Similar to A549-ICAM screen, HepG2 cells can be infected with retroviral vectors or transfected with siRNA, stimulated with various cytokines, and LDL receptor can be measured with FACs or by an LDL-binding assay (J Biol Chem 1993 Aug 15;268(23):17489-94, which is expressly incorporated herein by reference).

Day1:

Split cells HepG2 cells 4.5×10⁴ in a 24 well plate in the appropriate media and incubate at 37° C., 5% C02.

Day 2: Cells Should be 40-50% Confluent

siRNA

Transfect siRNA with oligofectamine. Pipet out media and replace with 500 uL of fresh media. Mix 3 uL of 20 uM siRNA duplexes with 50 uL of Optimem media. Add 3 uL of oligofectamine to 12 uL optimem. Wait 7-10 minutes. Combine the two solutions and pipet up and gently pipet up and down 3 times. Wait 20-25 minutes. Add 32 uL of optimem to adjust the volume to 100 uL. Add the entire mixture to the cells.

Retroviral

Infect cells using a standard spin infection protocol.

Day 3: Add 0.5 mL of Fresh Media

Day 4:

Wash cells in 1 mL PBS, remove PBS and add 100 uL of Trypsin/EDTA. 5 min later add 100 uL of fresh media. Pipet 4× up and down then transfer to a V-bottom 96 well plate. Spin down at 1200 rpm for 3 min. Resuspend in 200 uL of fresh media. Count representative wells by hemocytometer then compute the average cells/mi. Plate 1.5×10⁴ cells/well in a 96 well plate, the total final volume is 50 uL.

Day 5:

Add 50 uL of a 2× cytokine mixture. All cytokines can be purchased from Peprotech as a lyopholized powder.

Day 6: Detect LDL-Recptor Numbers With the LDL Binding Assay.

Rinse the cells 1×200 uL PBS and proceed with the binding assay as described previously (J Biol Chem 1993 Aug 15;268(23):17489-94).

### Example 4

### CHMC Low Cell Density IgE Activation: Tryptase and LTC4 Assays

Cultured human mast cells (CHMC) are obtained as described in U.S. Ser. No. 10/053,355, particularly at pages 46-50 which is expressly incorporated herein by reference. Screens for mast cell activation are performed as described below.

To duplicate 96-well U-bottom plates (Costar 3799) add 65 ul of compound dilutions or control samples that have been prepared in MT [137 mM NaCl, 2.7 mM KCl, 1.8 mM CaCl₂. 1.0 mM MgCl₂, 5.6 mM Glucose, 20 mM Hepes (pH 7.4), 0.1% Bovine Serum Albumin, (Sigma A4503)] containing 2% MeOH and 1% DMSO. Pellet CHMC cells (980 rpm, 10 min) and resuspend in pre-warmed MT. Add 65 ul of cells to each 96-well plate. Depending on the degranulation activity for each particular CHMC donor, load 1000-1500 cells/well. Mix four times followed by a 1 hr incubation at 37° C. During the 1 hr incubation, prepare 6× anti-IgE solution [rabbit anti-human IgE (1 mg/ml, Bethyl Laboratories A80-109A) diluted 1:167 in MT buffer]. Stimulate cells by adding 25 ul of 6X anti-IgE solution to the appropriate plates. Add 25 ul MT to un-stimulated control wells. Mix twice following addition of the anti-IgE. Incubate at 37° C. for 30 minutes. During the 30 minute incubation, dilute the 20 mM tryptase substrate stock solution [(Z-Ala-Lys-Arg-AMC 2TFA; Enzyme Systems Products, #AMC-246)] 1:2000 in tryptase assay buffer [0.1 M Hepes (pH 7.5), 10% w/v Glycerol, 10 uM Heparin (Sigma H4898) 0.01% NaN₃]. Spin plates at 1000 rpm for 10 min to pellet cells. Transfer 25 ul of supernatant to a 96-well black bottom plate and add 100 ul of freshly diluted tryptase substrate solution to each well. Incubate plates at room temperature for 30 min. Read the optical density of the plates at 355nm/460nm on a spectrophotometric plate reader.

Leukotriene C4 (LTC4) is also quantified using an ELISA kit on appropriately diluted supernatant samples (determined empirically for each donor cell population so that the sample measurement falls within the standard curve) following the supplier's instructions.

### Example 5

### CHMC High Cell Density IgE Activation: Degranulation (Tryptase, Histamine), Leukotriene (LTC4), and Cytokine (TNFalpha, IL-13) Assays

Cultured human mast cells (CHMC) are sensitized for 5 days with IL-4 (20 ng/ml), SCF (200 ng/ml), IL-6 (200 ng/ml), and Human IgE (CP 1035K from Cortx Biochem, 100-500ng/ml depending on generation) in CM medium. After sensitizing, cells are counted, pelleted (1000 rpm, 5-10 minutes), and resuspended at 1-2×10⁶ cells/ml in MT buffer. Add 100 ul of cell suspension to each well and 100 ul of compound dilutions. The final vehicle concentration is 0.5% DMSO. Incubate at 37° C. (5% CO₂) for 1 hour. After 1 hour of compound treatment, stimulate cells with 6× anti-IgE. Mix wells with the cells and allow plates to incubate at 37° C. (5% CO₂) for one hour. After 1 hour incubation, pellet cells (10 minutes, 1000 RPM) and collect 200 ul per well of the supernatant, being careful not to disturb pellet. Place the supernatant plate on ice. During the 7-hour step (see next) perform tryptase assay on supernatant that had been diluted 1:500. Resuspend cell pellet in 240 ul of CM media containing 0.5% DMSO and corresponding concentration of compound. Incubate CHMC cells for 7 hours at 37° C. (5% CO₂). After incubation, pellet cells (1000 RPM, 10 minutes) and collect 225 ul per well and place in −80° C. until ready to perform ELISAS. ELISAS are performed on appropriately diluted samples (determined empirically for each donor cell population so that the sample measurement falls within the standard curve) following the supplier's instructions.

### Example 6

### BMMC High Cell Density IgE Activation: Degranulation (Hexosiminidase, Histamine), Leukotriene (LTC4), and Cytokine (TNFalpha, IL-6) Assays

Preparation of WEHI-Conditioned Medium

WEHI-conditioned medium is obtained by growing murine myelomonocytic WEHI-3B cells (American Type Culture Collection, Rockville, Md.) in Iscove's Modified Eagles Media (Mediatech, Hernandon, Va.) supplemented with 10% heat-inactivated fetal bovine serum (FBS; JRH Biosciences, Kansas City, Mo.), 50 pM 2-mercaptoethanol (Sigma, St. Louis, Mo.) and 100 IU/mL penicillin-steptomycin (Mediatech) in a humidified 37° C., 5% CO₂/95% air incubator. An initial cell suspension is seeded at 200,000 cells/mL and then split 1:4 every 3-4 days over a period of two weeks. Cell-free supernatants are harvested, aliquoted and stored at −80° C. until needed.

Preparation of BMMC Medium

BMMC media consists of 20% WEHI-conditioned media, 10% heat-inactivated FBS (JHR Biosciences), 25 mM HEPES, pH7.4 (Sigma), 2mM L-glutamine (Mediatech), 0.1 mM nonessential amino acids (Mediatech), lmM sodium pyruvate (Mediatech), 50 zM 2mercaptoethanol (Sigma) and 100 IU/mL penicillin-streptomycin (Mediatech) in RPMI 1640 media (Mediatech). To prepare the BMMC Media, all components are added to a sterile IL filter unit and filtered through a 0.2 μm filter prior to use.

Protocol

Bone marrow derived mast cells (BMMC) are sensitized overnight with murine SCF (20 ng/ml) and monoclonal anti-DNP (10 ng/ml, Clone SPE-7, Sigma # D-8406) in BMMC media at a cell density of 666×10³ cells/ml. After sensitizing, cells are counted, pelleted (1000 rpm, 5-10 minutes), and resuspended at 1-3×10⁶ cells/ml in MT buffer. Add 100 ul of cell suspension to each well and 100 ul of compound dilutions. The final vehicle concentration is 0.5% DMSO. Incubate at 37° C. (5% CO₂) for 1 hour. After 1 hour of compound treatment, stimulate cells with 6× stimulus (60 ng/ml DNP-BSA). Mix wells with the cells and allow plates to incubate at 37° C. (5% CO₂) for one hour. After 1hour incubation, pellet cells (10 minutes, 1000 RPM) and collect 200 ul per well of the supernatant, being careful not to disturb pellet, and transfer to a clean tube or 96-well plate. Place the supernatant plate on ice. During the 4-5 hour step (see next) perform the hexosiminidase assay. Resuspend cell pellet in 240 ul WEI-conditioned media containing 0.5% DMSO and corresponding concentration of compound. Incubate BMMC cells for 4-5 hours at 37° C. (5% CO₂). After incubation, pellet cells (1000 RPM, 10 minutes) and collect 225 ul per well and place in −80° C. until ready to perform ELISAS. ELISAS are performed on appropriately diluted samples (determined empirically for each donor cell population so that the sample measurement falls within the standard curve) following the supplier's instructions.

Hexosaminidase assay: In a solid black 96-well assay plate, add 50 uL hexosaminidase substrate (4-methylumbelliferyl-N-acetyl-o-D-glucosaminide; 2 mM) to each well. Add 50 uL of BMMC cell supernatant (see above) to the hexoseaminidase substrate, place at 37° C. for 30 minutes and read the plate at 5, 10, 15, and 30 minutes on a spectrophotometer.

### Example 7

### Basophil IgE or Dustmite Activation: Histamine Release Assay (Watch Tense)

The basophil activation assay is carried out using whole human peripheral blood from donors allergic to dust mites with the majority of the red blood cells removed by dextran sedimentation. Human peripheral blood is mixed 1:1 with 3% dextran T500 and RBCs are allowed to settle for 20-25 min. The upper fraction is diluted with 3 volumes of D-PBS and cells are spun down for 10 min at 1500 rpm, RT. Supernatant is aspirated and cells are washed in an equal volume MT-buffer. Finally, cells are resuspended in MT-buffer containing 0.5% DMSO in the original blood volume. 80 uL cells are mixed with 20 uL compound in the presence of 0.5% DMSO, in triplicate, in a V-bottom 96-well tissue culture plate. A dose range of 8 compound concentrations is tested resulting in a 10-point dose response curve including maximum (stimulated) and minimum (unstimulated) response. Cells are incubated with compound for 1 hour at 37° C., 5% CO₂ after which 20 uL of 6× stimulus [1 ug/mL anti-IgE (Bethyl Laboratories) 667 au/mL house dustmite (Antigen Laboratories)] is added. The cells are stimulated for 30 minutes at 37° C., 5% CO₂. The plate is spun for 10 min at 1500 rpm at room temperature and 80 uL the supernatant is harvested for histamine content analysis using the histamine ELISA kit supplied by Immunotech. The ELISA is performed according to supplier's instructions.

### Example 8

### Monocyte Activation (Watch Tense)

This protocol measures cell surface markers of monocyte activation THP-1, U937 monocyte cell lines transfected with siRNA (see previous protocols) or infected with retroviral. Transfected or infected cells grown at 37° C. in 5% CO2 are stimulated with IFNY for either 3 days (U937) or 4 days (THP-1) cells in the appropriate growth media. The cells are treated with Nozyme to release them from the plate, then stained with various antibodies against CD11b, CD32, CD14, CD64, and HLA-DR conjugated to FITC, phycoerythrin (PE) or allophytin conugate (APC). As a control naive cells were stained and compared to stimulated cells.

### Example 9

### Osteoclast Differentiation Assay

This protocol is used to measure osteoclast differentiation in osteoclast precursors expressing a dominant negative mutant or siRNA. Differentiation is induced by treatment with TRANCE and M-CSF.

Mouse cells: From bone marrow, spleen, or the monocytic cell line RAW264.7: Mouse bone marrow cells or spleen cells are cultured in a-MEM (Life Technologies, Grand Island, N.Y.) containing 10% FBS with M-CSF (5 ng/ml) for 12 h in 100-mm diameter dishes (Corning, Glass, Corning, N.Y.; 1×10⁷ cells/10 ml/dish) to separate adherent cells and nonadherent cells. Then, nonadherent cells are harvested and cultured with M-CSF (30 ng/ml) in 100-mm diameter dishes (1×1 cells/10 ml/dish). After 2 days of culture, floating cells are removed and attached cells are used as osteoclast precursors. To generate osteoclasts, osteoclast precursors are cultured with TRANCE (300 ng/ml) and M-CSF (30 ng/ml) for 3 days in 96-well culture plates (Corning; 2×104 cells/0.2 ml/well) or in 60-mm diameter dishes (Corning; 2.5×106 cells/5 ml/dish). To purify mature osteoclasts, cells are treated with cell dissociation solution (Sigma-Aldrich) for 5 min, and the sides of the plates are tapped. Most mononuclear cells are detached after tapping, but multinucleated osteoclasts remained attached to the culture plates. To generate osteoclasts from the murine myeloid RAW264.7 cell line (American Type Culture Collection, Manassas, VA), cells are cultured in 96-well culture plates (1×103 cells/0.2 ml/well) with TRANCE (300 ng/ml)for4 days. Old media are replaced with fresh media containing TRANCE (300 ng/ml) on day 3. To generate human osteoclasts, freshly isolated human peripheral blood monocytes are cultured in 96-well culture plates (5×104 cells/0.2 ml/well) with TRANCE (300 ng/ml) and M-CSF (30 ng/ml) for 5 days. Old media are replaced with fresh media containing TRANCE (300 ng/ml) and M-CSF (30 ng/ml) on day 3. In some experiments, indicated concentration of PGN, poly(l:C) RNA, LPS, or CpG DNA is added to the cultures with or without TRANCE and M-CSF. All cells are cultured at 37° C. and 5% CO2.

Osteoclast formation is measured by a tartrate-resistant acid phosphatase (TRAP) solution assay or TRAP staining as described (Mol Cell. 1999 December;4(6):1041-9, Nature. 2002 Jul 25;418(6896):443-7).

For human cells: THP-1cells, human PBMC, human CD14+PBMC, U937 cells, human bone marrow.

Osteolcast differentiation is induced by treating the cells in the appropriate media with recombinant soluble TRANCE (10-100 ng/mL) and M-CSF (10-100 ng/mL) as described (Calcif Tissue lnt. 1998 Jun;62(6):527-31). Fresh media and cytokines are added every 3-4 days. Typically multinucleated giant cells are produced in 5 days—3 weeks. Osteoclast formation is measured by a tartrate-resistant acid phosphatase (TRAP) solution assay or TRAP staining as described (Mol Cell. 1999 Dec;4(6):1041-9, Nature. 2002 Jul 25;418(6896):443-7).

### Example 10

Following staining, as described below, the cells are analyzed using the methods described in U.S. Ser No. - - - - - - (attorney docket no. RIGL-016-00US), filed Aug. 28, 2002.

HCS PAD ASSAY—Fix and Dapi Stain Procedure

Using Hudson Plate Crane, Bio-Tek Elx405 plate washer, and Labsystems Multidrop 384

Plates should be Packard View black 96-well plates #6005182, clear plate seals #6005185 PBS—calcium & magnesium-free Cellgro cat # 21-040-CM Supplies: plate seals, marker, 20 uL pipettman & tips, 5 mL tube and holder, conical 500 mL flasks & holder, timer, 1 mg/mL DAPI stock

1. Make fix and warm

Fix is 7.4% formaldehyde in PBS MUST BE PRE-WARMED TO 37° C.

| | | To_mL warm PBS, |
|---|---|---|
| | Add_mL of 10% | then place in incubator to |
| Number of plates | formaldehyde stock | warm |
| 1 plate | 7.4 mL | 2.6 mL |
| 12 (round up to 15) | 111 | 39 |
| 24 (round up to 30) | 222 | 78 |

| | | | Then add this |
|---|---|---|---|
| | Add_uL of | | mixture to_mL |
| Number of | 1 mg/mL | | PBS just before use, |
| plates | DAPI stock | To_mL DW | shake immediately |
| 12 plates | 18 uL | 7.2 mL | 300 mL |

HCS_FIX method on robot removes media down to 100 uL, then adds 100 uL of fix, FAC: 3.7% formaldehyde. Make fix for 10 mL per plate, plus 6 mL dead vol. in Multidrop tubing. Make Dapi int. stock in DW
HCS_DAPI method removes wash down to 30 uL, then adds 170 uL of DAPI per well, FAC = 0.5 ng/mL DAPI in PBS. Make DAPI for 17 mL per plate, plus 6 mL dead volume Empty robot's waste bottle and rinse Put fresh PBS in correct bottle, transfer drawing tube and prime the system full of PBS

5. SET MULTIDROP TO 100 uL, 96 well plate and 12 columns and PRIME the Multidrop with formaldehyde

6. Take plates out of incubator and stack with flange facing inward, label w/bar code

7. RUN HCS_FIX and 5_TO_{—}4, START TIMER COUNTDOWN FROM 30 MIN when fix goes on the first plate

8. At 30 minute mark, if have 12 plates, set methods for:

HCS_WASH

5_TO_{—}4

HCS_DAPI

5_TO_{—}4 (if less than 12, stop here & time 15 minutes from DAPI onto first plate)

HCS_WASH

5_TO_{—}4

HCS_WASH

As the wash begins, CHANGE MULTIDROP TO 170 uL, rinse tubing and PRIME with DAPI

CLEANUP

1. Seal plates and store in frig

2. Empty waste bottle and rinse

3. Transfer drawing tube to water bottle and prime the system full of water

4. Clean and remove Multidrop tubing and place in drawer, reset Multidrop to 100 uL

Fixative:

Polysciences, Inc. Cat# 04018,1 liter, 10% formaldehyde (methanol-free) ultrapure EM grade

DAPI:

Molecular Probes D-1306 10 mg

Dilute to 5mg/mL in DW, keep in frig. Make lmg/mL stock in DW and store in fig for 3 months

TABLE 7
Summary of siRNA PAD data

| | siRNA PAD (Cell cycle arrest |
|---|---|
| Gene | in) |
| E2.1 | |
| E2.15 | |
| E2.2 | S |
| E2.16 | |
| E2.17 | |
| E2.3 | NE |
| E2.4 | G2 |
| E2.18 | |
| E2.5 | NE |
| E2.19 | |
| UBE2D3 | NE |
| Hs 1 SNP | |
| E2.20 | |
| E2.21 | |
| E2.6 | NE |
| E2.22 | |
| E2.23 | |
| E2.7 | NE |
| E2.8 | — |
| E2.9 | G2 |
| E2.10 | G2/M |
| E2.24 | |
| E2.11 | |
| E2.12 | NE |
| E2.13 | |
| E2.25 | |
| E2.14 | NE |

### Example 11

### Dissociated Spinal Cord Cultures

Primary cultures of dissociated spinal cord and DRGs are prepared as described by Roy et al. (1998). In brief, spinal cords and associated ganglia are dissected from embryos, dissociated with trypsin, and plated on 12-mm coverslips precoated with poly-D-lysine and extracellular matrix (Sigma-Aldrich) at a density of 2.5×105 cells per well of a four-well plate (Nunclon). Approximately 1-2×106 cells are obtained from each spinal cord, each cord being processed and plated separately. For microinjection studies, cultures are prepared from embryos and plated at a density of 6.5×105 per well in 12-well dishes (Roy et al., 1998). All cells are plated in modified N3 medium as described in Roy et al. (1998). On days 4 and 5, cultures are treated with 1 μM cytosine arabinoside for 1-2 d to limit growth of nonneuronal cells, and are maintained in modified N3 medium at 37° C. in 5% CO². Cultures are used for analyses after 14 d in vitro studies and after 4-6 wk for microinjection studies.

### Example 12

### DRG Neuron-dissociated Spinal Cord Cocultures

DRG cultures are prepared as described in O'Ferrall et al. (2000) with the following modifications. The medium for plating and general maintenance is as for the dissociated spinal cord cultures described above. DRG neurons are plated at 12-15 dissociated DRGs per well of a four-well plate containing coverslips precoated as above.

For coculture experiments, Falcon cell culture inserts (0.4 μM polyethylene terephthalate track etched membrane, six-well format; Becton Dickinson) are placed in six-well insert companion plates that contained medium only, or that had been preplated with dissociated spinal cord cultures at a density of 106 cells per well. DRG neurons are plated on glass coverslips as described above and allowed to establish for 4 d. Coverslips are then transferred to Falcon cell culture inserts and cocultured with the dissociated spinal cord cultures or with medium only for 10-14 d. After this time, coverslips are removed and labeled using the TUNEL assay as a marker of apoptosis.

Immunocytochemistry

Immunocytochemistry is performed as in Roy et al. (1998) using antibodies from Chemicon (peripherin, monoclonal MAB1527, and polyclonal AB1515; poylclonal neurofilament antibodies to NF-L, AB1983; NF-M, AB1981; and neurofilament heavy subunit [NF-H], AB1982; all 1:1,000), Sigma-Aldrich (monoclonal antibodies to neurofilaments NF-L, NR4; NF-M, NN18; NF-H, N52; and -tubulin, DM1A; all 1:1,000), and nuclear envelope breakdown (polyclonal antibody to activated caspase-3, 1:100; following supplier recommendations). Antibody distribution is visualized by epifluorescence/confocal microscopy after incubation with the appropriate secondary antibody (Alexa Fluor-labeled secondary antibody; 1:100; Molecular Probes).

For electron microscopy and immunohistochemical analysis of transgenic mouse tissue sections, the method of Beaulieu et al. (1999) is used.

Immunoblotting

Cells are harvested in 7 mM Tris, pH 6.75, containing 2% SDS and 10% glycerol, and assayed for total protein using the bicinchoninic acid assay. Loadings of 10-15 pg of protein are routinely analyzed on 6-12% gradient SDS-polyacrylamide gels and then blotted to polyvinyldifluoride membrane. For immunoblotting, membranes are incubated with monoclonal antibodies recognizing peripherin (MAB1527, 1:5,000; Chemicon) or actin (MAB1501,1:10,000; Chemicon), and antibody binding is revealed using the ECL detection system (NEN Life Sciences).

TUNEL Assays

The In Situ Cell Death Detection Kit, POD, from Roche Molecular Diagnostics (Laval, QC) is used for TUNEL assays, with DAB as the substrate (Gavrieli et al., 1992). Fluorescent double labeling of cultures with antibody to peripherin is performed in conjunction with the TUNEL assay to enable correlation of TUNEL-positive cells with the presence of peripherin aggregates. TUNEL labeling in itself is not indicative of apoptosis, and confirmatory evidence of apoptosis is obtained from morphological criteria such as cell shrinkage and maintenance of an intact plasma membrane, chromatin condensation, clearly observed with DAB-TUNEL labeling and labeling with antibody recognizing activated caspase-3 (Wyllie, 1980; Majno and Joris, 1995; Thornberry and Lazebnik, 1998; Nijhawan et al., 2000). TUNEL-positive DRG neurons from dissociated spinal cord cultures are counted after 14 and 21 d in culture. To calculate the percentage of TUNEL-positive DRG neurons, cell cultures are counted using the 25× objective covering ten fields in the vertical axis and ten in the horizontal axis. Individual cultures are counted a minimum of three times and each time no less than 100 DRG neurons are counted. The percentage specific apoptosis (% experimental apoptosis—% spontaneous apoptosis/100 —% spontaneous apoptosis) is calculated using the averages of the total counts from Per and WT cultures from the same litter. This enables a direct comparison between different culturing experiments.

### Example 13

### Cell Cycle Analysis With BrdU

Cells (A549, Hela) were plated 24 hours before transfection on 24-well plate (Costar) in 500 □I growth media supplemented with 10% FBS.

.siRNA were obtained from Dharmacon Inc. or Xeragon. Inc.

60 pmol of siRNA duplex is mixed with 50 μl of Opti-Mem media (Gibco). In another tube 3 μl of Oligofectamine Reagent (Invitrogen) is mixed with 12 μl of Opti-Mem media and incubated 10 min at room temperature. Solutions are combined and incubated 25 min at room 10 temperature. Then 32 μl of fresh of Opti-Mem media is added to final volume of 100 μl. The 100 μl of siRNA- Oligofectamine mix is added to the cells. 16 hours after transfection cells are ished 2 times with PBS, trypsinized and plated on 6 well plate with density 2500 cells/cm² for Cell Cycle analysis with BrdU and FACScan instrument or 1500 cells per well onto 96 well tissue clture plate (Costar) for PAD assay with Cellomics instrument.

72 hours after transfection BrdU was added at concentration 10 μ. 4 hours after incubation with BrdU cells were collected, fixed and prepared for Cell Cycle analysis as it was described before (Kastan et al., 1991, Cancer research, 51: 6304-6311; White et al., 1994, Genes and Development 8: 666-677; Serrano et al, 1997, Cell, 88(5):593-602, which are expressly incorporated herein by reference). Cell cycle analysis was performed using a Becton Dickinson FACScan instrument.

TABLE 8
Summary of Cell Cycle Assay Results

| Gene (with siRNA) | Cell cycle arrest in: |
|---|---|
| E1.1 | G1 and G2/M; apoptosis |
| E1.4 | G2/M; apoptosis |
| E1.2 | NE |
| E1.3 | G2/M; apoptosis |
| E1.5 | NE |
| E1.6 | G1; G2/M |
| E1.7 | G1 |
| E1.8 | G1; G2/M |
| E1.9 | ND |
| E1.10 | NE |
| E1.11 | G2/M, apoptosis |
| E1.12 | NE |
| E1.13 | NE |
| E1.14 | G2/M |

TABLE 9
Summary of Cell Cycle Assay Results

| Gene (with siRNA) | Cell Cycle arrest in: |
|---|---|
| E2.1 | |
| E2.15 | |
| E2.2 | G2/M, apoptosis |
| E2.16 | |
| E2.17 | |
| E2.3 | NE |
| E2.4 | G2/M |
| E2.18 | |
| E2.5 | NE |
| E2.19 | |
| UBE2D3 | NE |
| E2.20 | |
| E2.21 | |
| E2.6 | G2/M |
| E2.22 | |
| E2.23 | |
| E2.7 | NE |
| E2.8 | NE |
| E2.9 | G2/M |
| E2.10 | G2/M |
| E2.24 | |
| E2.11 | |
| E2.12 | G2/M |
| E2.13 | |
| E2.25 | |
| E2.14 | NE |

TABLE 10
Summary of Cell Cycle Assay Results

| Gene (with siRNA) | Cell Cycle (arrest in): |
|---|---|
| E3.4 | G2 |
| E3.5 | G2 |
| E3.1 | S, G2 |
| E3.3 | ND |

### Example 14

### GFP Cell Tracker

CellTtracker™ assays were performed as described in the Molecular Probes catalog, as is well understood in the art. Cells also co-expressed variant ubiquitin agents. Results of experiments with E2 variants are summarized below.

TABLE 11
Summary of Cell Tracker Results

| | HeLa DN | |
|---|---|---|
| Gene | GFP/CeIl Tracker | A549 DN GFP/CT |
| E2.1 | NE | |
| E2.15 | NE | |
| E2.2 | NE | |
| E2.16 | NE | |
| E2.17 | NE | |
| E2.3 | NE | |
| E2.4 | NE | |
| E2.18 | NE | |
| E2.5 | | |
| E2.19 | NE | |
| UBE2D3 | NE | |
| E2.20 | NE | |
| E2.21 | NE | |
| E2.6 | NE | |
| E2.22 | NE | |
| E2.23 | INH | |
| E2.7 | | |
| E2.8 | NE | |
| E2.9 | NE | |
| E2.10 | NE | |
| E2.24 | | |
| E2.11 | | |
| E2.12 | 374%/1.4 | 38%/1.3 |
| E2.13 | | |
| E2.25 | NE | 6%/0.8 |
| E2.14 | | |

1. A method comprising:
a) contacting a cell with a negative effector of a ubiquitin agent, said ubiquitin agent being selected from the group consisting of a ubiquitin moiety, a ubiquitin activating agent (E1), a ubiquitin conjugating agent (E2) and a ubiquitin ligating agent (E3);
b) screening said cell for an altered phenotype, whereby said ubiquitin agent is identified as a modulator of said phenotype.

2. The method of claim 1, wherein said contacting comprises introducing a nucleic acid into said cell.

3. The method of claim 2, wherein said nucleic acid is said negative effector of said uniquitin agent.

4. The method of claim 3, wherein said nucleic acid is an siRNA targeted against mRNA encoding said ubiquitin agent.

5. The method of claim 3, wherein said nucleic acid is antisense to an mRNA or gene encoding said ubiquitin agent.

6. The method of claim 2, wherein said nucleic acid comprises a sequence encoding said negative effector of said ubiquitin agent.

7. The method of claim 6, wherein said nucleic acid is in the form of an expression construct comprising a promoter, operably linked to said sequence encoding said negative effector.

8. The method of claim 7, wherein said expression construct is contained within a vector.

9. The method of claim 8, wherein said vector is a retroviral vector.

10. The method of claim 6, wherein said negative effector is selected from the group consisting of an siRNA targeted against mRNA encoding said ubiquitin agent, nucleic acid antisense to an mRNA or gene encoding said ubiquitin agent, a dominant negative variant of said ubiquitin agent.

11. The method of claim 1, wherein said altered phenotype is altered cell cycle regulation.

12. The method of claim 1, wherein said altered phenotype is altered cellular proliferation and/or altered cell viability.

13. The method of claim 1, wherein said altered phenotype is altered response to an inflammatory cytokine.

14. The method of claim 1, wherein said cell is a T cell and said altered phenotype is altered response to a T cell activating agent.

15. The method of claim 1, wherein said cell is a B cell and said altered phenotype is altered response to a B cell activating agent.

16. The method of claim 1, wherein said cell is an endothelial cell and said altered phenotype is altered response to an angiogenesis stimulating agent.

17. The method of claim 1, wherein said altered phenotype is altered chemotaxis and/or haplotaxis.

18. The method of claim 1, wherein said cell is a mast cell and said altered phenotype is altered response to mast cell activation.

19. The method of claim 1, wherein said altered phenotype is altered exocytosis.

20. The method of claim 1, wherein said altered phenotype is altered release or synthesis of LDL.

21. The method of claim 1, wherein said altered phenotype is altered response to a signaling agent.
